(19) United States
(12) Patent Application Publication
(10) Pub. No.: US 2024/0016965 A1
(43) Pub. Date: Jan. 18, 2024

(54) ULTRAVIOLET-SENSING MEMBER AND ULTRAVIOLET-SENSING KIT

(71) Applicant: FUJIFILM Corporation, Tokyo (JP)

(72) Inventor: Nobutaka FUKAGAWA, Shizuoka (JP)

(73) Assignee: FUJIFILM Corporation, Tokyo (JP)

(21) Appl. No.: 18/472,251

(22) Filed: Sep. 22, 2023

Related U.S. Application Data
(63) Continuation of application No. PCT/JP2022/011657, filed on Mar. 15, 2022.

(30) Foreign Application Priority Data
Mar. 31, 2021 (JP) 2021-059771

(51) Int. Cl. A61L 2/28; C07D 307/91

(52) U.S. Cl. CPC A61L 2/28; C07D 307/91

(57) ABSTRACT

The present invention provides an ultraviolet-sensing member in which it is easy to determine whether an irradiation amount which inactivates the novel coronavirus has been irradiated, and an ultraviolet-sensing kit. The ultraviolet-sensing member of the present invention contains a sensitizer having an absorption maximal wavelength in a range of 200 to 230 nm, a photoactivator, and a color-forming agent.

### CROSS-REFERENCE TO RELATED APPLICATIONS

This application is a Continuation of PCT International Application No. PCT/JP2022/011657 filed on Mar. 15, 2022, which claims priority under 35 U.S.C. § 119(a) to Japanese Patent Application No. 2021-059771 filed on Mar. 31, 2021. The above applications are hereby expressly incorporated by reference, in their entirety, into the present application.

### BACKGROUND OF THE INVENTION

### 1. Field of the Invention

The present invention relates to an ultraviolet-sensing member and an ultraviolet-sensing kit.

### 2. Description of the Related Art

A measurement of an amount of ultraviolet irradiation has been carried out in various fields. Specific examples thereof include a measurement of an amount of ultraviolet irradiation to an object to be irradiated in a curing reaction of an ultraviolet curing resin, and a measurement of an amount of ultraviolet irradiation to an object to be irradiated in an ultraviolet sterilization of food or the like.

As the measurement of the amount of ultraviolet irradiation, for example, JP2015-191001A discloses a method of using “UV label” (UV-H manufactured by NiGK Corporation), and WO2017/158943A discloses a method of using “UV scale” (manufactured by FUJIFILM Corporation).

On the other hand, in recent years, infection with novel coronavirus (COVID-19) has been a major social problem.

Under these circumstances, in Hiroki, Kitagawa et al., “Effectiveness of 222-nm ultraviolet light on disinfecting SARS-CoV-2 surface contamination”, American Journal of Infection Control, Internet (https://www.sciencedirect.com/science/article/pii/S0196655320308099), an inactivation effect of the novel coronavirus using ultraviolet rays with a wavelength of 222 nm has been reported. More specifically, in Hiroki, Kitagawa et al., “Effectiveness of 222-nm ultraviolet light on disinfecting SARS-CoV-2 surface contamination”, American Journal of Infection Control, Internet (https://www.sciencedirect.com/science/article/pii/S0196655320308099), it has been reported that 99.7% of the novel coronavirus is inactivated by radiating ultraviolet rays with a wavelength of 222 nm at an illuminance of 0.1 mW/cm² for 30 seconds.

### SUMMARY OF THE INVENTION

As disclosed in Hiroki, Kitagawa et al., “Effectiveness of 222-nm ultraviolet light on disinfecting SARS-CoV-2 surface contamination”, American Journal of Infection Control, Internet (https://www.sciencedirect.com/science/article/pii/S0196655320308099), since ultraviolet rays with a wavelength of 222 nm are effective in inactivating the novel coronavirus, for example, by irradiating ultraviolet rays with a wavelength of 222 nm to a member which is touched by an unspecified number of people, such as a doorknob and a touch panel, it is possible to prevent the infection with the novel coronavirus. In this case, it is desirable that it is possible to easily measure whether or not an irradiation amount of ultraviolet rays which achieves the inactivation of the novel coronavirus is applied to a predetermined position.

In a case of measuring, using a known UV label and UV scale in the related art, whether the amount of light with a wavelength of 222 nm, which inactivates the novel coronavirus infection, is irradiated, the present inventors cannot determine the irradiation amount because almost no change in tint is observed with the UV label and UV scale. More specifically, even in a case where the UV label and the UV scale are irradiated with ultraviolet rays with a wavelength of 222 nm at an integrated illuminance of 3 mJ/cm², almost no change in tint is observed.

In view of the above-described circumstances, an object according to a first embodiment of the present invention is to provide an ultraviolet-sensing member in which it is easy to determine whether an irradiation amount which inactivates the novel coronavirus has been irradiated.

An object according to a second embodiment of the present invention is to provide an ultraviolet-sensing member in which it is easy to determine whether an irradiation amount which inactivates the novel coronavirus has been irradiated, and basis for determination can be stored stably for a certain period of time.

Another object of the present invention is to provide an ultraviolet-sensing kit.

As a result of intensive studies to achieve the above-described objects, the present inventors have found that the above-described objects can be achieved by the following configurations, and have completed the present invention.
<1>

An ultraviolet-sensing member comprising:
a sensitizer having an absorption maximal wavelength in a range of 200 to 230 nm;
a photoactivator; and
a color-forming agent.
<2>

The ultraviolet-sensing member according to <1>,
in which the sensitizer has a naphthalene structure.
<3>

The ultraviolet-sensing member according to <1> or <2>,
in which the photoactivator has a halomethyl-s-triazine structure.
<4>

The ultraviolet-sensing member according to any one of <1> to <3>,
in which the color-forming agent has a spirolactone structure represented by Formula (I).

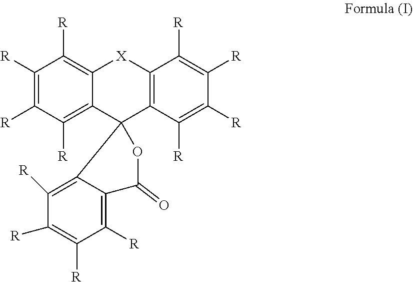

In Formula (I), X represents an oxygen atom, a sulfur atom, or —NR19-, where R19 represents a hydrogen atom, an alkyl group, or an aryl group, and R's each independently represent a hydrogen atom or a monovalent substituent.
<5>

An ultraviolet-sensing member,
in which, in a case where, using a KrCl excimer lamp as a light source, the ultraviolet-sensing member is irradiated with light through a filter which substantially shields light having a wavelength of 230 to 300 nm until an irradiation amount of light having a wavelength of 222 nm reaches 3 mJ/cm²,
   using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and
   a value of a cyan optical density of the ultraviolet-sensing member before the light irradiation is denoted as C1, a value of a cyan optical density of the ultraviolet-sensing member after 2 hours from the light irradiation is denoted as C2, a value of a cyan optical density of the ultraviolet-sensing member after 24 hours from the light irradiation is denoted as C3, a value of a yellow optical density of the ultraviolet-sensing member before the light irradiation is denoted as Y1, a value of a yellow optical density of the ultraviolet-sensing member after 2 hours from the light irradiation is denoted as Y2, a value of a yellow optical density of the ultraviolet-sensing member after 24 hours from the light irradiation is denoted as Y3, a value of a magenta optical density of the ultraviolet-sensing member before the light irradiation is denoted as M1, a value of a magenta optical density of the ultraviolet-sensing member after 2 hours from the light irradiation is denoted as M2, and a value of a magenta optical density of the ultraviolet-sensing member after 24 hours from the light irradiation is denoted as M3,
any one of a difference between C1 and C2, a difference between Y1 and Y2, or a difference between M1 and M2 is 0.20 or more, and
a rate of change in optical density of a color which exhibits a largest difference among the difference between C1 and C2, the difference between Y1 and Y2, and the difference between M1 and M2, represented by the following expression (II), is −50% or more and 50% or less,

rate of change in optical density (%)=[(optical density after 24 hours of irradiation)−(optical density after 2 hours of irradiation)]//[(optical density after 2 hours of irradiation)−(optical density before irradiation)]×100. Expression (II)
<6>

The ultraviolet-sensing member according to any one of <1> to <4>,
in which a difference between C1 and C2, a difference between Y1 and Y2, a difference between M1 and M2, and a rate of change in optical density satisfy the requirements of <5>.
<7>

The ultraviolet-sensing member according to any one of <1> to <6>, in which the ultraviolet-sensing member is a sheet-like ultraviolet-sensing member.
<8>

An ultraviolet-sensing kit comprising:
the ultraviolet-sensing member according to any one of <1> to <7>.
<9>

An ultraviolet-sensing kit comprising:
the ultraviolet-sensing member according to any one of <1> to <7>, in which, in a case where the ultraviolet-sensing member is irradiated with light until an irradiation amount of light having a wavelength of 313 nm reaches 9 mJ/cm²,
   using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and
   a value of a cyan optical density of the ultraviolet-sensing member before the light irradiation is denoted as C4, a value of a cyan optical density of the ultraviolet-sensing member after 2 hours from the light irradiation is denoted as C5, a value of a yellow optical density of the ultraviolet-sensing member before the light irradiation is denoted as Y4, a value of a yellow optical density of the ultraviolet-sensing member after 2 hours of light irradiation is denoted as Y5, a value of a magenta optical density of the ultraviolet-sensing member before the light irradiation is denoted as M4, and a value of a magenta optical density of the ultraviolet-sensing member after 2 hours from the light irradiation is denoted as M5,
all of a difference between C4 and C5, a difference between Y4 and Y5, and a difference between M4 and M5 are 0.15 or less.

According to the first embodiment of the present invention, it is possible to provide an ultraviolet-sensing member in which it is easy to determine whether an irradiation amount which inactivates the novel coronavirus has been irradiated.

According to the second embodiment of the present invention, it is possible to provide an ultraviolet-sensing member in which it is easy to determine whether an irradiation amount which inactivates the novel coronavirus has been irradiated, and basis for determination can be stored stably for a certain period of time.

According to the present invention, it is possible to provide an ultraviolet-sensing kit.

### BRIEF DESCRIPTION OF THE DRAWINGS

FIG. 1 is a schematic cross-sectional view showing an example of a first embodiment of the ultraviolet-sensing member according to the present invention.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

In the present specification, the numerical ranges shown using “to” indicate ranges including the numerical values described before and after “to” as the lower limit value and the upper limit value.

In addition, regarding numerical ranges that are described stepwise in the present specification, an upper limit value or a lower limit value described in a numerical range may be replaced with an upper limit value or a lower limit value of another stepwise numerical range. In addition, in the numerical range described in the present specification, an upper limit value and a lower limit value described in a certain numerical range may be replaced with values shown in Examples.

In addition, in the present specification, a solid content means a component forming a composition layer formed of a composition, and in a case where the composition contains a solvent (for example, organic solvent, water, and the like), the solid content means all components excluding the solvent. In addition, in a case where the components are components which form a composition layer, the components are considered to be solid contents even in a case where the components are liquid components.

In addition, in the present specification, ultraviolet rays mean light having a wavelength range of 10 to 400 nm.

In addition, in the present specification, (meth)acrylic means “at least one of acrylic or methacrylic”.

In addition, in the present specification, “boiling point” means a boiling point at a standard atmospheric present.

### First Embodiment

The ultraviolet-sensing member according to the first embodiment of the present invention contains a sensitizer having an absorption maximal wavelength in a range of 200 to 230 nm, a photoactivator, and a color-forming agent.

As will be described later, the ultraviolet-sensing member preferably has an aspect of including a support and an ultraviolet-sensing layer, and in this case, it is preferable that the ultraviolet-sensing layer contains a sensitizer having an absorption maximal wavelength in a range of 200 to 230 nm, a photoactivator, and a color-forming agent.

Hereinafter, each material contained in the ultraviolet-sensing member (or the ultraviolet-sensing layer) will be described in detail.

(Color-Forming Agent)

The ultraviolet-sensing member according to the first embodiment contains a color-forming agent. Among these, the ultraviolet-sensing member preferably includes an ultraviolet-sensing layer containing a color-forming agent. A specific configuration of the ultraviolet-sensing member will be described in detail later.

Here, the “color-forming agent” refers to a compound which forms color from a state of being substantially colorless (a state in which it is colorless or exhibits a light color) by action of acid, oxidation, light irradiation, or the like.

The type of the color-forming agent is not particularly limited, and examples thereof include a color-forming agent which forms color by being oxidized, a color-forming agent which forms color by action of acid, and a color-forming agent which forms color by action of light. Among these, a color-forming agent which forms color by being oxidized or a color-forming agent which forms color by action of acid is preferable, and a color-forming agent which forms color by action of acid is more preferable.

As the color-forming agent, a leuco coloring agent or a photochromic coloring agent is preferable, and a leuco coloring agent is more preferable.

The photochromic coloring agent is known as a compound which forms color by being isomerized by the action of light, a compound which forms color by progressing a ring-closing reaction by the action of light, a compound which forms color by progressing a ring-opening reaction by the action of light, or the like, and a known photochromic coloring agent can be used. It is preferable that the photochromic coloring agent has a coloring-decolorizing reaction which reversibly proceeds with energy.

The above-described leuco coloring agent is preferably a compound which forms color in a case of being oxidized from a substantially colorless state (hereinafter, also referred to as “oxidative color-forming leuco coloring agent”) or a compound which forms color by the action of acid from a substantially colorless state (hereinafter, also referred to as “acid color-forming leuco coloring agent”).

Examples of the leuco coloring agent include a triarylmethanephthalide-based compound, a fluoran-based compound, a phenothiazine-based compound, an indolylphthalide-based compound, an azaindolylphthalide-based compound, a leuco auramine-based compound, a rhodamine lactam-based compound, a triarylmethane-based compound, a diarylmethane-based compound, a triazene-based compound, a spiropyran-based compound, a thiazine-based compound, and a fluorene-based compound.

For details of the above-described compounds, reference can be made to the description of U.S. Pat. No. 3,445,234A, JP1993-257272A (JP-H5-257272A), and paragraphs 0029 to 0034 of WO2009/008248A.

The color-forming agent may be used alone or in combination of two or more kinds thereof.

Oxidative Color-Forming Leuco Coloring Agent

As one aspect of the oxidative color-forming leuco coloring agent, a compound having one or two hydrogen atoms, which forms color by removing electrons, is preferable. Examples of such an oxidative color-forming leuco coloring agent include (a) aminotriarylmethane, (b) aminoxanthine, (c) aminothioxanthine, (d) amino-9,10-dihydroacridine, (e) aminophenoxazine, (f) aminophenothiazine, (g) aminodihydrophenazine, (h) aminodiphenylmethane, (i) leuco indamine, (j) aminohydrocinnamic acid (cyanethane and leuco methine), (k) hydrazine, (l) leuco indigoid dye, (m) amino-2,3-dihydroanthraquinone, (n) tetrahalo-p,p′-biphenol, (o) 2-(p-hydroxyphenyl)-4,5-diphenylimidazole, and (p) phenethylaniline, which are described in U.S. Pat. No. 3,445,234A. Among the above-described (a) to (p), (a) to (i) form color by losing one hydrogen atom, and (j) to (p) form color by losing two hydrogen atoms.

Among these, aminoarylmethane is preferable, and aminotriarylmethane is more preferable.

As the aminotriarylmethane, a compound represented by Formula (L) or an acid salt thereof is preferable.

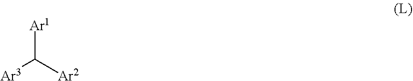

In the formula, Ar¹ represents (A1) a phenyl group having a R¹R²N-substituent at a para position with respect to a bond to a methane carbon atom specified in the formula. Ar² represents (A1) a phenyl group having a R¹R²N-substituent at a para position with respect to a bond to a methane carbon atom specified in the formula or (A2) a phenyl group having, at an ortho position with respect to the methane carbon atom specified in the formula, a substituent selected from the group consisting of an alkyl group (preferably, an alkyl group having 1 to 4 carbon atoms), an alkoxy group (preferably, an alkoxy group having 1 to 4 carbon atoms), a fluorine atom, a chlorine atom, and a bromine atom. R¹ and R² each independently represent a hydrogen atom, an alkyl group having 1 to 10 carbon atoms, a 2-hydroxyethyl group, a 2-cyanoethyl group, or a benzyl group.

Ar³ represents the same group as at least one of Ar¹ or Ar², or a group different from Ar¹ and Ar². In a case where Ar³ represents a group different from Ar1 and Ar², Ar³ represents (B1) a phenyl group which may be substituted with a substituent selected from the group consisting of a lower alkyl group (preferably, an alkyl group having 1 to 4 carbon atoms), a lower alkoxy group (preferably, an alkoxy group having 1 to 4 carbon atoms), a chlorine atom, a diphenylamino group, a cyano group, a nitro group, a hydroxy group, a fluorine atom, a bromine atom, an alkylthio group, an arylthio group, a thioester group, an alkylsulfonic acid group, an arylsulfonic acid group, a sulfonic acid group, a sulfonamide group, an alkylamide group, and an arylamide group; (B2) a naphthyl group which may be substituted with a substituent selected from the group consisting of an amine group, a di-lower alkylamino group, and an alkylamino group; (B3) a pyridyl group which may be substituted with an alkyl group; (B4) a quinolyl group; or (B5) an indolinylidene group which may be substituted with an alkyl group.

In Formula (L), R¹ and R² are each independently preferably a hydrogen atom or an alkyl having 1 to 4 carbon atoms.

In addition, in Formula (L), it is preferable that all of Ar¹, Ar², and Ar³ are (A1) the phenyl group having a R¹R²N-substituent at a para position with respect to a bond to a methane carbon atom specified in the formula, and it is more preferable that all of Ar¹, Ar², and Ar³ are the same group thereof.

Specific examples of the oxidative color-forming leuco coloring agent include tris(4-dimethylaminophenyl)methane, tris(4-diethylaminophenyl)methane, bis(4-diethylaminophenyl)-(4-diethylamino-2-methylphenyl)methane, bis(4-diethylamino-2-methylphenyl)-(4-diethylaminophenyl)methane, bis(1-ethyl-2-methylindol-3-yl)-phenylmethane, 2-N-(3-trifluoromethylphenyl)-N-ethylamino-6-diethylamino-9-(2-methoxycarbonylphenyl)xanthene, 2-(2-chlorophenyl)amino-6-dibutylamino-9-(2-methoxycarbonylphenyl)xanthene, 2-dibenzylamino-6-diethylamino-9-(2-methoxycarbonylphenyl)xanthene, benzo[a]-6-N,N-diethylamino-9,2-methoxycarbonylphenylxanthene, 2-(2-chlorophenyl)-amino-6-dibutylamino-9-(2-methylphenylcarboxamidophenyl)xanthene, 3,6-dimethoxy-9-(2-methoxycarbonyl)-phenylxanthene, benzoyl leuco methylene blue, and 3,7-bis-diethylaminophenoxazine.

Acid Color-Forming Leuco Coloring Agent

As one aspect of the acid color-forming leuco coloring agent, a compound which forms color by donating electrons or receiving protons such as an acid is preferable. Specific examples thereof include a compound which has a partial skeleton such as lactone, lactam, sultone, spiropyrane, ester, and amide, in which these partial skeletons are ring-opened or cleaved upon contact with an acid or a proton.

Examples of the leuco coloring agent which forms color by the action of acid (acid color-forming leuco coloring agent) include 3,3-bis(2-methyl-1-octyl-3-indolyl)phthalide, 6′-(dibutylamino)-2′-bromo-3′-methylspiro[phthalido-3,9′-xanthene], 3-(4-diethylamino-2-ethoxyphenyl)-3-(1-ethyl-2-methylindol-3-yl)-4-azaphthalide, 3-(4-diethylamino-2-ethoxyphenyl)-3-(1-n-octyl-2-methylindol-3-yl)phthalide, 3-[2,2-bis(1-ethyl-2-methylindol-3-yl)vinyl]-3-(4-diethylaminophenyl)-phthalide, 2-anilino-6-dibutylamino-3-methylfluorane, 6-diethylamino-3-methyl-2-(2,6-xylidino)-fluorane, 2-(2-chloroanilino)-6-dibutylaminofluorane, 3,3-bis(4-dimethylaminophenyl)-6-dimethylaminophthalide, 2-anilino-6-diethylamino-3-methylfluorane, 9-[ethyl(3-methylbutyl)amino]spiro[12H-benzo[a]xanthene-12,1′(3′H)isobenzofuran]-3′-one, 2′-methyl-6′-(N-p-tolyl-N-ethylamino)spiro[isobenzofuran-1(3H),9′-[9H]xanthene]-3-one, 3′,6′-bis(diethylamino)-2-(4-nitrophenyl)spiro[isoindole-1,9′-xanthene]-3-one, 9-(N-ethyl-N-isopentylamino)spiro[benzo[a]xanthene-12,3′-phthalide], 2′-anilino-6′-(N-ethyl-N-isopentylamino)-3′-methylspiro[phthalide-3,9′-[9H]xanthene], and 6′-(diethylamino)-1′,3′-dimethylfluorane.

In addition, from the viewpoint of color formability, a spirolactone compound represented by Formula (I) is preferable as the acid color-forming agent of the present invention.

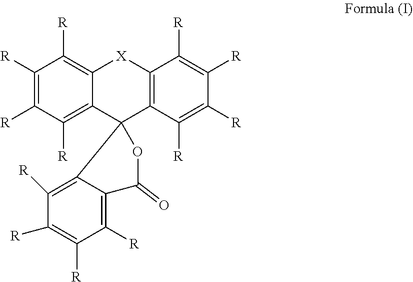

In Formula (I), X represents an oxygen atom, a sulfur atom, or —NR19-, where R19 represents a hydrogen atom, an alkyl group, or an aryl group. R's each independently represent a hydrogen atom or a monovalent substituent.

In Formula (I), the alkyl group represented by R19 is preferably an alkyl group having 1 to 30 carbon atoms, more preferably an alkyl group having 1 to 15 carbon atoms, and still more preferably an alkyl group having 1 to 10 carbon atoms. The above-described alkyl group may be linear or branched, or may have a ring structure. Specific examples thereof include a methyl group, an ethyl group, a propyl group, a butyl group, a pentyl group, a hexyl group, a heptyl group, an octyl group, a nonyl group, a decyl group, an undecyl group, a dodecyl group, a tridecyl group, a hexadecyl group, an octadecyl group, an eicosyl group, an isopropyl group, an isobutyl group, an s-butyl group, a tert-butyl group, an isopentyl group, a neopentyl group, a 1-methylbutyl group, an isohexyl group, a 2-ethylhexyl group, a 2-methylhexyl group, a cyclohexyl group, a cyclopentyl group, and a 2-norbornyl group. Among these alkyl groups, a methyl group, an ethyl group, a propyl group, or a butyl group is preferable.

The above-described alkyl group may have a substituent. Examples of the substituent include an alkoxy group, an aryloxy group, an amino group, an alkylthio group, an arylthio group, a halogen atom, a carboxy group, a carboxylate group, a sulfo group, a sulfonate group, an alkyloxycarbonyl group, an aryloxycarbonyl group, and a group obtained by combining these groups.

As the aryl group represented by R19, an aryl group having 6 to 30 carbon atoms is preferable, an aryl group having 6 to 20 carbon atoms is more preferable, and an aryl group having 6 to 12 carbon atoms is still more preferable.

The above-described aryl group may have a substituent. Examples of the substituent include an alkyl group, an alkoxy group, an aryloxy group, an amino group, an alkylthio group, an arylthio group, a halogen atom, a carboxy group, a carboxylate group, a sulfo group, a sulfonate group, an alkyloxycarbonyl group, an aryloxycarbonyl group, and a group obtained by combining these groups.

Specific examples thereof include a phenyl group, a naphthyl group, a p-tolyl group, a p-chlorophenyl group, a p-fluorophenyl group, a p-methoxyphenyl group, a p-dimethylaminophenyl group, a p-methylthiophenyl group, and a p-phenylthiophenyl group.

Among these aryl groups, a phenyl group, a p-methoxyphenyl group, a p-dimethylaminophenyl group, or a naphthyl group is preferable.

Examples of the monovalent substituent represented by R include an alkyl group, an alkoxy group, an aryl group, an aryloxy group, an amino group, an alkylthio group, an arylthio group, a halogen atom, a carboxyl group, a carboxylate group, a sulfo group, a sulfonate group, an alkyloxycarbonyl group, and an aryloxycarbonyl group. Here, as the alkyl group and the aryl group included in the monovalent substituent, the description of the alkyl group and the aryl group in R19 above can be referred to.

In Formula (I), from the viewpoint of color forming efficiency, X is preferably an oxygen atom.

Specific examples of the compound which functions as the acid color-forming agent are shown below, but the present invention is not limited thereto.

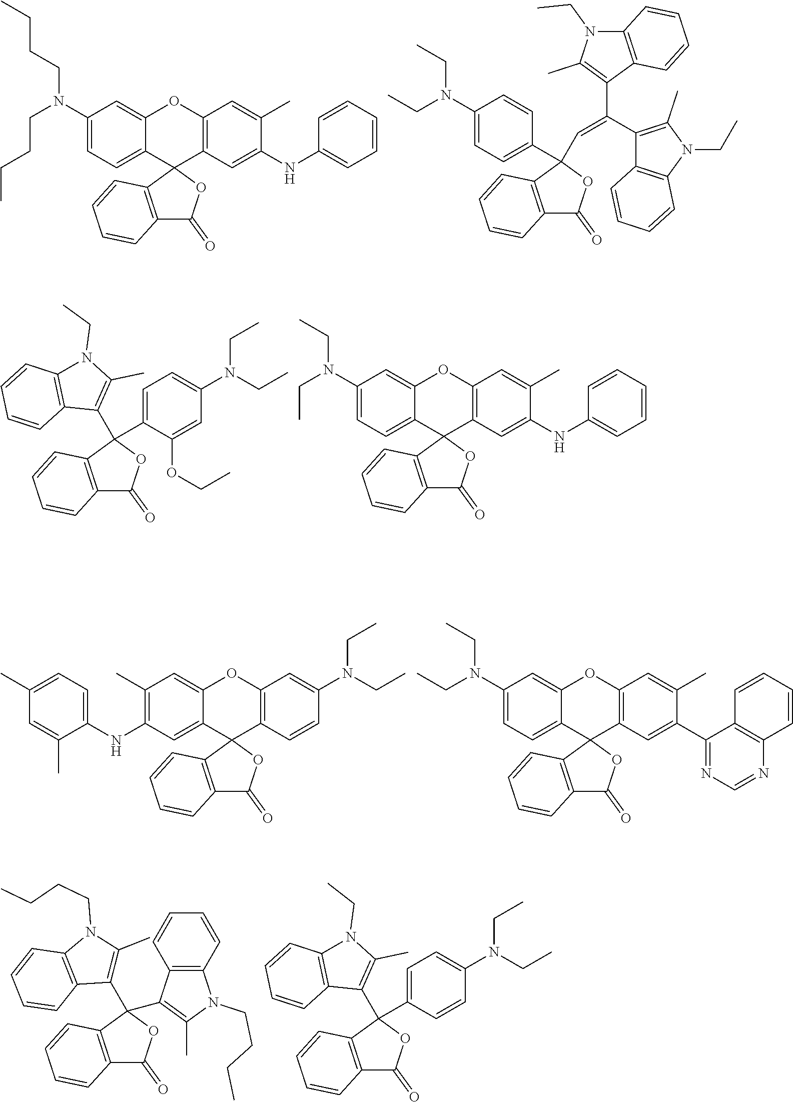

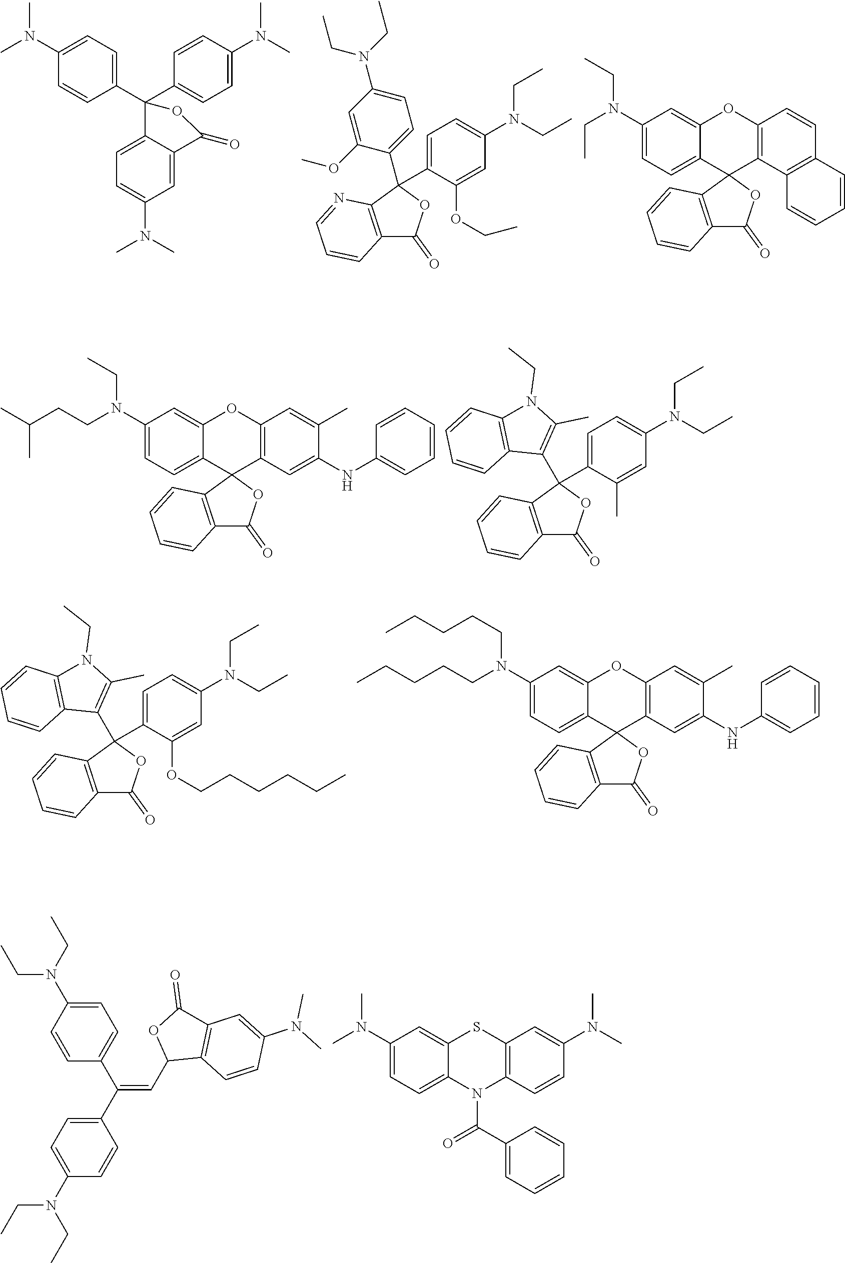

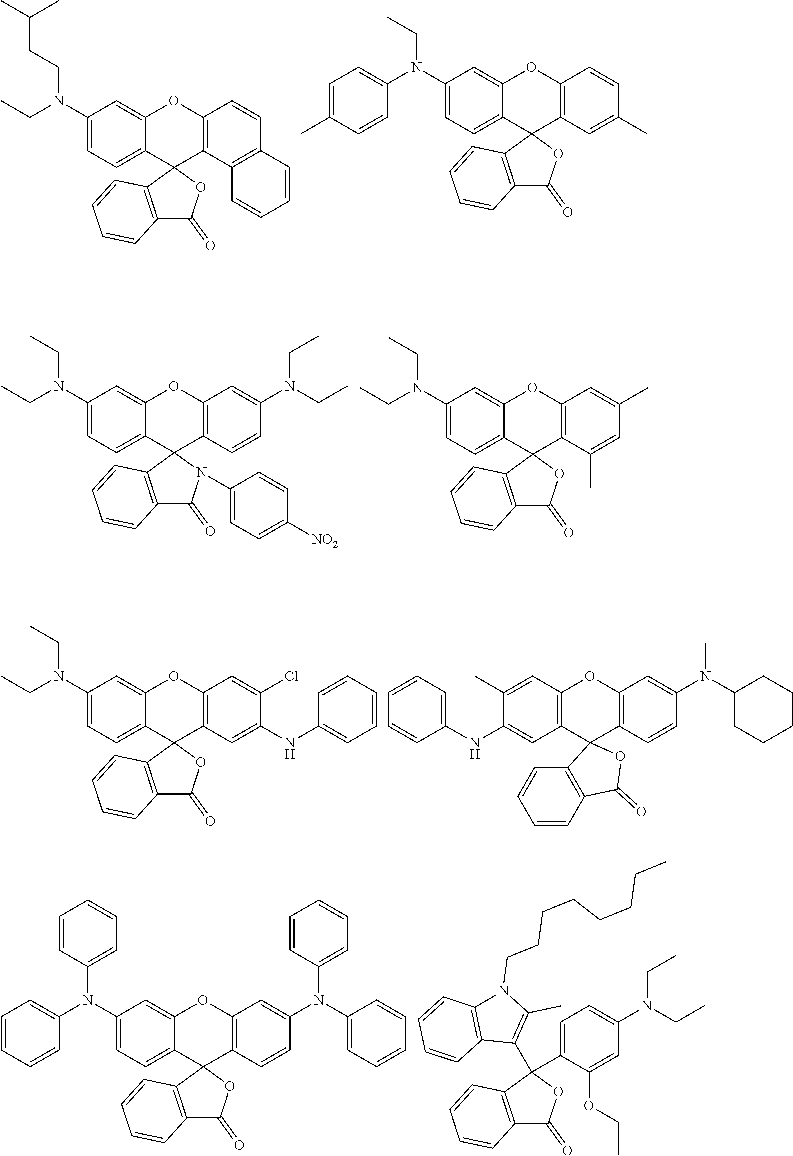

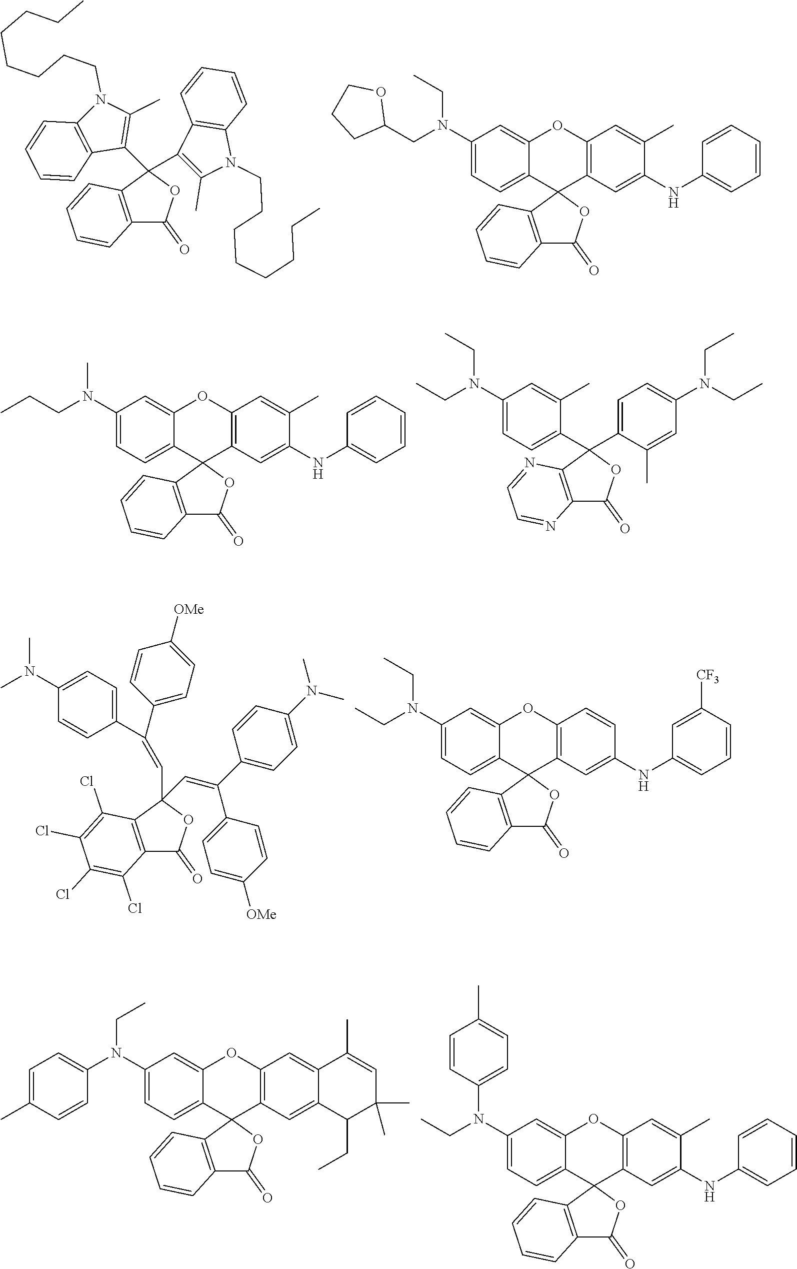

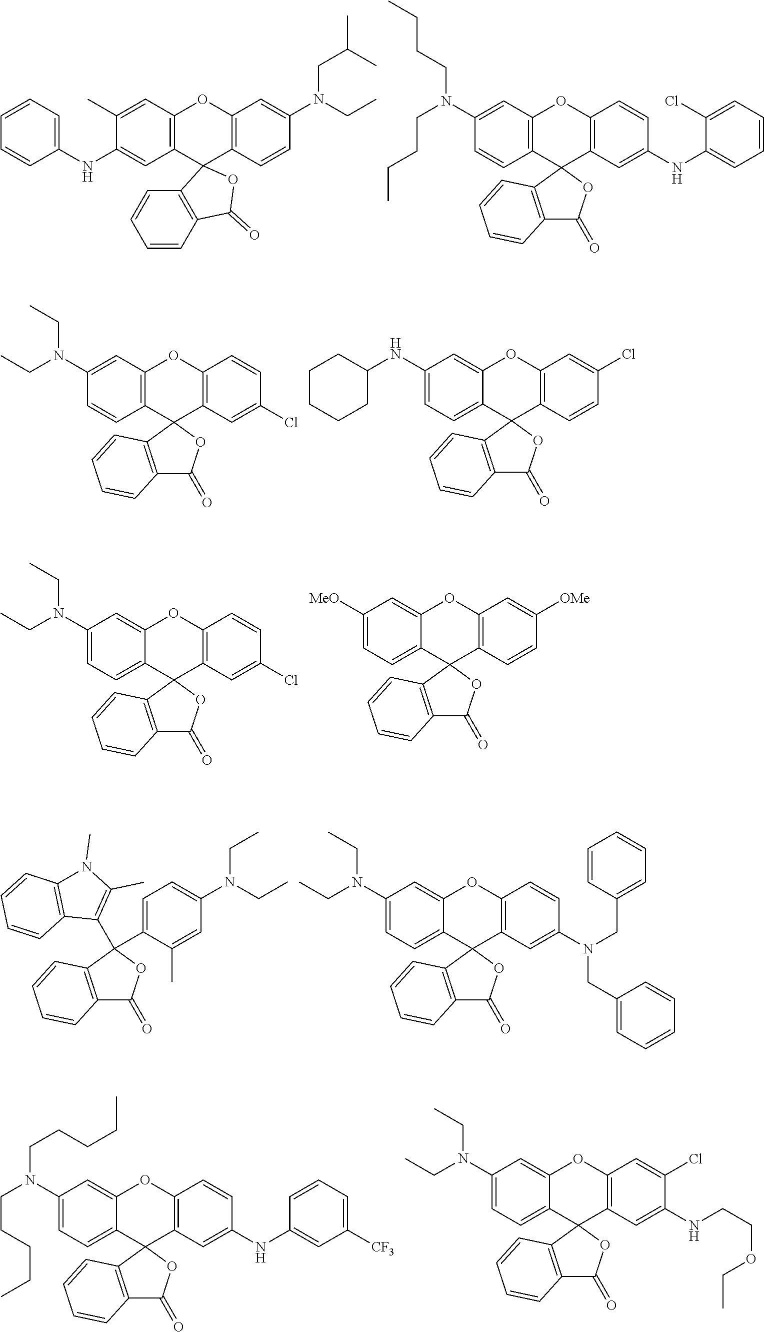

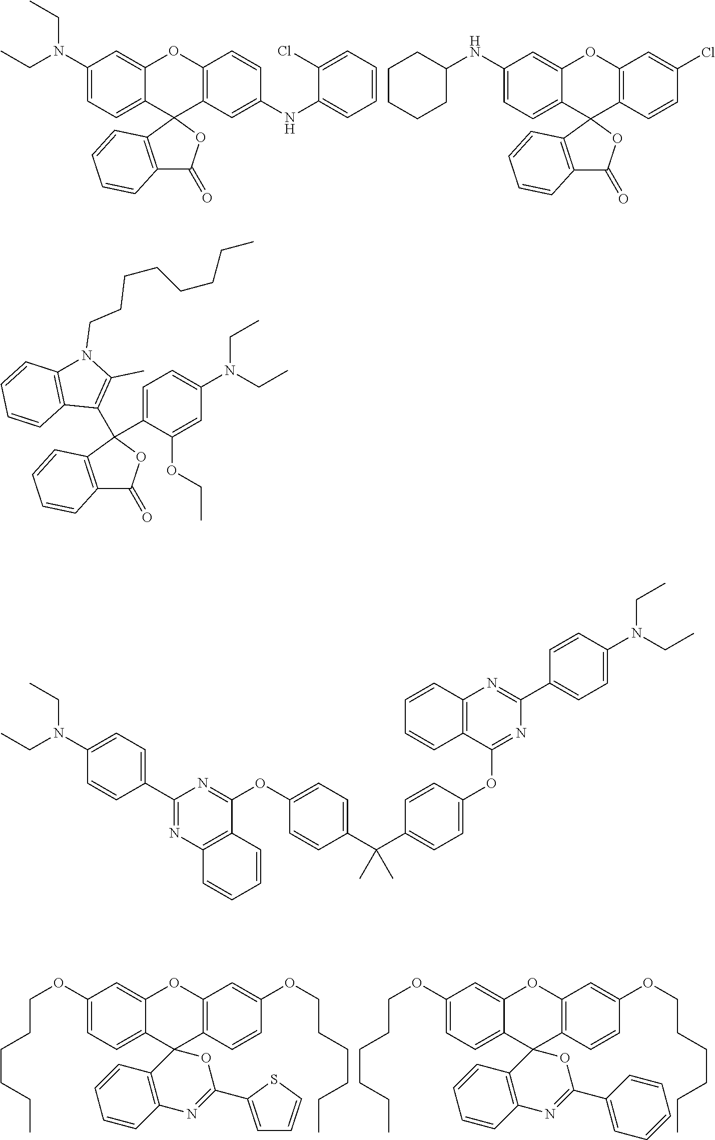

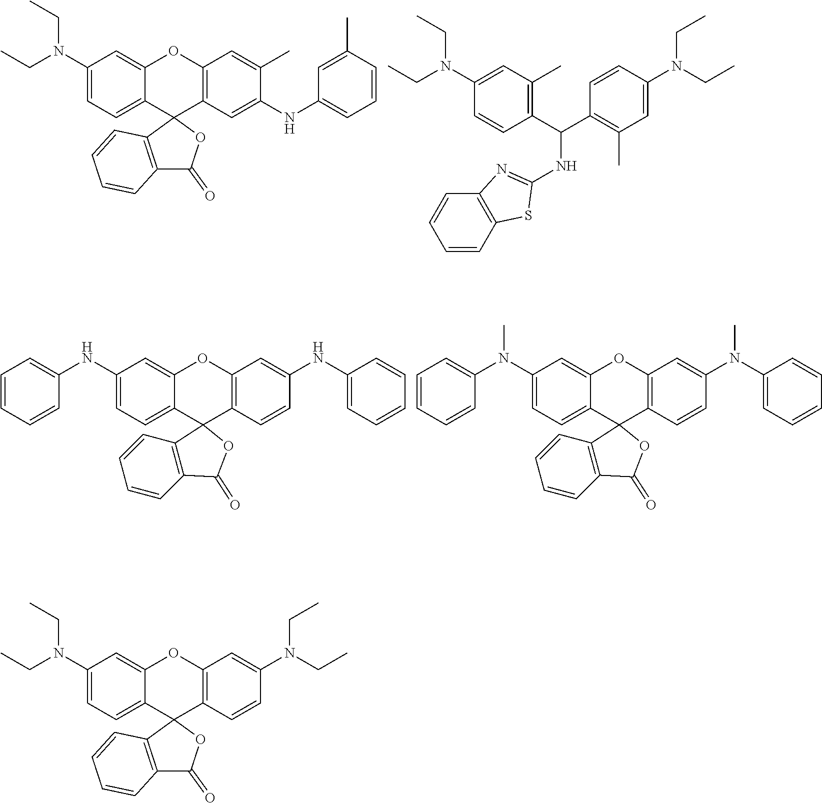

A molecular weight of the compound represented by General Formula (I) is not particularly limited, but is preferably 300 or more and more preferably 350 or more. The upper limit thereof is not particularly limited, but is preferably 2,000 or less and more preferably 1,000 or less.

A content of the color-forming agent in the ultraviolet-sensing layer is not particularly limited, but from the viewpoint that it is easier to determine whether the irradiation amount which inactivates the novel coronavirus has been irradiated (hereinafter, also simply referred to as “viewpoint that the effect of the present invention is more excellent”), the content of the color-forming agent per unit area (m²) of the ultraviolet-sensing layer is preferably 0.500 g/m² or less, more preferably 0.300 g/m² or less, and still more preferably 0.140 g/m² or less. The lower limit thereof is not particularly limited, but is preferably 0.010 g/m² or more and more preferably 0.030 g/m² or more. By setting the content of the color-forming agent in the ultraviolet-sensing laver within the above-described range, since excessive absorption of 222 nm by the color-forming agent is suppressed, the minimum amount of color-forming agent required for the color-forming reaction can be used, and as a result, it is presumed that the difference in optical density in the second embodiment can be set to 0.20 or more.

The above-described content of the color-forming agent can be calculated by cutting out an area having many ultraviolet-sensing layers from the ultraviolet-sensing member, immersing the ultraviolet-sensing layers in acetonitrile for 2 days, and then analyzing the obtained solvent by liquid chromatography. The acetonitrile is prevented from volatilizing during the immersion. As necessary, a calibration curve of the content of the color-forming agent to be detected may be created before the measurement of the liquid chromatography.
Measurement conditions of the liquid chromatography are as follows.
Equipment: Nexera manufactured by Shimadzu Corporation
Column: Capcell pak C18 UG-120
Eluent: water/acetonitrile
Oven: 40° C.
Injection: 5 μL
Detection: maximum absorption wavelength of color-forming agent to be detected
Flow Rate: 0.2 mL/min

In addition, the content of the color-forming agent in the ultraviolet-sensing layer is preferably 0.1 to 20 parts by mass, more preferably 0.3 to 10 parts by mass, and still more preferably 0.5 to 3 parts by mass with respect to 100 parts by mass of the ultraviolet-sensing layer.

(Photoactivator)

The ultraviolet-sensing member according to the first embodiment contains a photoactivator. Among these, it is preferable that the ultraviolet-sensing layer included in the ultraviolet-sensing member contains a photoactivator.

The photoactivator is not particularly limited as long as it is a compound which is activated by the photoactivator itself absorbing light or a compound which is activated by accepting electrons, holes, or energy from another compound absorbing light, but the photoactivator activated by light preferably acts on the color-forming agent to form color, and is preferably a compound activated by ultraviolet rays. The photoactivator is preferably any one or more of a photooxidant or a photoacid generator. In a case where the ultraviolet-sensing member contains the color-forming agent which forms color by being oxidized, the photoactivator preferably includes a photooxidant, and in a case where the ultraviolet-sensing member contains the color-forming agent which forms color by action of acid, the photoactivator preferably includes a photoacid generator.

From the viewpoint that the effect of the present invention is more excellent, a mass ratio of a content of the photoactivator to a content of the color-forming agent (photoactivator/color-forming agent (mass ratio)) is preferably more than 1.00 and more preferably 3.00 or more. The upper limit thereof is not particularly limited, but is preferably 40.00 or less, more preferably 30.00 or less, still more preferably 25.00 or less, particularly preferably 20.00 or less, and most preferably 10.00 or less. By setting the mass ratio of the content of the photoactivator to the content of the color-forming agent within the above-described range, since excessive absorption of 222 nm by the color-forming agent is suppressed, the photoactivator or the sensitizer efficiently absorbs 222 nm, and as a result, it is presumed that the color-forming reaction proceeds efficiently and the difference in optical density of the second embodiment described later can be set to 0.20 or more.

The above-described mass ratio of the content of the photoactivator to the content of the color-forming agent can be measured by liquid chromatography after methanol extraction in the same manner as the above-described content of the color-forming agent. The photoactivator is detected at the maximum absorption wavelength of the photoactivator to be detected, the color-forming agent is detected at the maximum absorption wavelength of the color-forming agent to be detected, and the mass ratio thereof is obtained.

Photooxidant

The photooxidant is preferably a compound which can cause the forming of the color-forming agent by being activated by ultraviolet rays to generate a radical and exhibit an action of extracting the hydrogen atom of the color-forming agent.

Among these, the photooxidant is preferably one or more of a radical generator and an organic halogen compound. It is also preferable to use the radical generator and the organic halogen compound in combination as the photoacid generator. In a case where the radical generator and the organic halogen compound are used in combination, from the viewpoint that the gradation properties of the colored part are more excellent, a content ratio (radical generator/organic halogen compound (mass ratio)) of the radical generator to the organic halogen compound is preferably 0.1 to 10 and more preferably 0.5 to 5.

Radical Generator

The radical generator is not particularly limited as long as it is a compound which is activated by ultraviolet rays to generate a radical.

As the radical generator, a hydrogen-extracting radical generator is preferable. The hydrogen-extracting radical generator exhibits an action of extracting hydrogen atoms from the color-forming agent to promote the oxidation of the color-forming agent.

Examples of the radical generator include azide polymers described in The Lecture Summary, p. 55 for the Spring Meeting of the Society of Photographic Science and Technology of Japan, 1968; azide compounds described in U.S. Pat. No. 3,282,693A, such as 2-azidobenzoxazole, benzoylazide, and 2-azidobenzimidazole; compounds described in U.S. Pat. No. 3,615,568A, such as 3′-ethyl-1-methoxy-2-pyridothiacyanine perchlorate, 1-methoxy-2-methylpyridinium, and p-toluenesulfonate; lophine dimer compounds described in JP1987-039728B (JP-S62-039728B), such as a 2,4,5-triarylimidazole dimer; benzophenone; p-aminophenyl ketone; polynuclear quinone; and thioxanthene.

Among these, one or more selected from a lophine dimer and benzophenone is preferable, and a lophine dimer is more preferable.

Examples of the lophine dimer include a hexaarylbiimidazole compound. As the hexaarylbiimidazole-based compound, compounds described in paragraph 0047 of WO2016/017701A can be referred to, the contents of which are incorporated in the present specification.

Among these, 2,2′-bis(2-chlorophenyl)-4,4′,5,5′-tetraphenyl-1,2′-biimidazole is preferable. As the 2,2′-bis(2-chlorophenyl)-4,4′,5,5′-tetraphenyl-1,2′-biimidazole, for example, “B-IMD” (manufactured by KUROGANE KASEI Co., Ltd.) and “B-CIM” (manufactured by Hodogaya Chemical Co., Ltd.) can be used.

As the lophine dimer, a compound represented by General Formula (1) is also preferable.

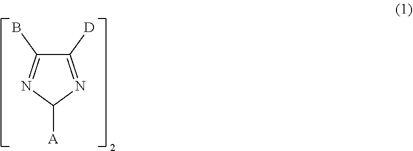

In the formula, A, B, and D each independently represent a carbocyclic or heteroaryl group, which is unsubstituted or substituted with a substituent which does not interfere with dissociation of the dimer to an imidazolyl group or the oxidation of the color-forming agent.

It is preferable that B and D are each independently unsubstituted or have 1 to 3 substituents, and it is preferable that A is unsubstituted or has 1 to 4 substituents.

As the compound represented by General Formula (1) and a method of preparing the compound, a finding known as the lophine dimer or the like can be utilized. For example, the description of column 4, line 22 and column 6, line 3 of U.S. Pat. No. 3,552,973A can be referred to, the contents of which are incorporated in the present specification.

The radical generator may be used alone or in combination of two or more kinds thereof.

Organic Halogen Compound

The organic halogen compound can promote the oxidation of the color-forming agent.

From the viewpoint that the gradation properties of the colored part are more excellent, the organic halogen compound is preferably a compound in which the number of halogen atoms in the molecule is 3 or more. The upper limit value of the number of halogen atoms is preferably 9 or less. The organic halogen compound is a compound other than the lophine dimer and the benzophenone.

The organic halogen compound may be used alone or in combination of two or more kinds thereof.

Examples of the organic halogen compound include compounds represented by General Formulae (2) to (7).

P⁰—CX₃ (2)

In the formula, P⁰ represents a hydrogen atom, a halogen atom, an alkyl group which may have a substituent, or an aryl group which may have a substituent. X's each independently represent a halogen atom.

Examples of the halogen atom represented by P⁰ and X include a fluorine atom, a chlorine atom, a bromine atom, and an iodine atom, and a chlorine atom or a bromine atom is preferable.

Examples of the substituent which can be included in the alkyl group and aryl group represented by P⁰ include a hydroxy group, a halogen atom, an alkyl group having 1 to 6 carbon atoms, a haloalkyl group having 1 to 6 carbon atoms, an acetyl group, and an alkoxy group having 1 to 6 carbon atoms.

Examples of the compound represented by General Formula (2) include trichloromethane, tribromomethane, carbon tetrachloride, carbon tetrabromide, p-nitrobenzotribromide, bromotrichloromethane, benzotrichloride, hexabromoethane, iodoform, 1,1,1-tribromo-2-methyl-2-propanol, 1,1,2,2-tetrabromoethane, 2,2,2-tribromoethanol, and 1,1,1-trichloro-2-methyl-2-propanol.

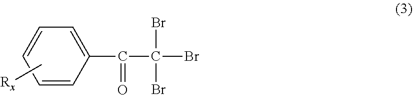

In the formula, R represents a substituent. x represents an integer of 0 to 5.

Examples of the substituent represented by R include a nitro group, a halogen atom, an alkyl group having 1 to 3 carbon atoms, a haloalkyl group having 1 to 3 carbon atoms, an acetyl group, a haloacetyl group, and an alkoxy group having 1 to 3 carbon atoms.

In a case where a plurality of R's are present in the formula, the R's may be the same or different from each other.

x is preferably 0 to 3.

Examples of the compound represented by General Formula (3) include o-nitro-α,α,α-tribromoacetophenone, m-nitro-α,α,α-tribromoacetophenone, p-nitro-α,α,α-tribromoacetophenone, α,α,α-tribromoacetophenone, and α,α,α-tribromo-3,4-dichloroacetophenone.

R¹—SO₂—X¹ (4)

In the formula, R¹ represents an alkyl group which may have a substituent or an aryl group which may have a substituent. X¹ represents a halogen atom.

As the alkyl group represented by R¹, an alkyl group having 1 to 20 carbon atoms is preferable, an alkyl group having 1 to 10 carbon atoms is more preferable, and an alkyl group having 1 to 6 carbon atoms is still more preferable.

As the aryl group represented by R¹, an aryl group having 6 to 20 carbon atoms is preferable, an aryl group having 6 to 14 carbon atoms is more preferable, and an aryl group having 6 to 10 carbon atoms is still more preferable.

Examples of the substituent which can be included in the alkyl group and aryl group represented by R¹ include a nitro group, a halogen atom, an alkyl group having 1 to 3 carbon atoms, a haloalkyl group having 1 to 3 carbon atoms, an acetyl group, a haloacetyl group, and an alkoxy group having 1 to 3 carbon atoms.

Examples of the halogen atom represented by X¹ include a fluorine atom, a chlorine atom, a bromine atom, and an iodine atom, and a chlorine atom, a bromine atom, or an iodine atom is preferable and a chlorine atom or a bromine atom is more preferable.

Examples of the compound represented by General Formula (4) include 2,4-dinitrobenzenesulfonyl chloride, o-nitrobenzenesulfonyl chloride, m-nitrobenzenesulfonyl chloride, 3,3′-diphenylsulfonedisulfonyl chloride, ethanesulfonyl chloride, p-bromobenzenesulfonyl chloride, p-nitrobenzenesulfonyl chloride, p-3-benzenesulfonyl chloride, p-acetamidobenzenesulfonyl chloride, p-chlorobenzenesulfonyl chloride, p-toluenesulfonyl chloride, methanesulfonyl chloride, and benzenesulfonyl bromide.

R²—S—X² (5)

In the formula, R² represents an alkyl group which may have a substituent or an aryl group which may have a substituent. X² represents a halogen atom.

The alkyl group which may have a substituent and the aryl group which may have a substituent, represented by R², are the same as those of R¹ in General Formula (4), and suitable aspects thereof are also the same.

Examples of the halogen atom represented by X² include a fluorine atom, a chlorine atom, a bromine atom, and an iodine atom, and a chlorine atom, a bromine atom, or an iodine atom is preferable and a chlorine atom or a bromine atom is more preferable.

Examples of the compound represented by General Formula (5) include 2,4-dinitrobenzenesulfenyl chloride and o-nitrobenzenesulfenyl chloride.

R³-L¹-CX³X⁴X⁵ (6)

In the formula, R³ represents an aryl group which may have a substituent or a heteroaryl group which may have a substituent. L¹ represents —SO— or —SO₂—. X³, X⁴, and X⁵ each independently represent a hydrogen atom or a halogen atom. However, all of X³, X⁴, and X⁵ are not hydrogen atoms at the same time.

As the aryl group represented by R³, an aryl group having 6 to 20 carbon atoms is preferable, an aryl group having 6 to 14 carbon atoms is more preferable, and an aryl group having 6 to 10 carbon atoms is still more preferable.

As the heteroaryl group represented by R³, a heteroaryl group having 4 to 20 carbon atoms is preferable, a heteroaryl group having 4 to 13 carbon atoms is more preferable, and a heteroaryl group having 4 to 9 carbon atoms is still more preferable.

Examples of the substituent which can be included in the aryl group and heteroaryl group represented by R³ include a nitro group, a halogen atom, an alkyl group having 1 to 3 carbon atoms, a haloalkyl group having 1 to 3 carbon atoms, an acetyl group, a haloacetyl group, and an alkoxy group having 1 to 3 carbon atoms.

Examples of the halogen atom represented by X³, X⁴, and X⁵ include a fluorine atom, a chlorine atom, a bromine atom, and an iodine atom, and a chlorine atom, a bromine atom, or an iodine atom is preferable and a chlorine atom or a bromine atom is more preferable.

Examples of the compound represented by General Formula (6) include hexabromodimethyl sulfoxide, pentabromodimethyl sulfoxide, hexabromodimethylsulfone, trichloromethylphenylsulfone, tribromomethylphenylsulfone (BMPS), trichloro-p-chlorophenylsulfone, tribromomethyl-p-nitrophenylsulfone, 2-trichloromethylbenzothiazolesulfone, 4,6-dimethylpyrimidine-2-tribromomethylsulfone, tetrabromodimethylsulfone, 2,4-dichlorophenyl-trichloromethylsulfone, 2-methyl-4-chlorophenyltrichloromethylsulfone, 2,5-dimethyl-4-chlorophenyltrichloromethylsulfone, 2,4-dichlorophenyltrimethylsulfone, and tri-p-tolylsulfonium trifluoromethanesulfonate. Among these, trichloromethylphenylsulfone or tribromomethylphenylsulfone (BMPS) is preferable.

R⁴CX⁶X⁷X⁸ (7)

In the formula, R⁴ represents a heteroaryl group which may have a substituent. X⁶, X⁷, and X⁸ each independently represent a hydrogen atom or a halogen atom. However, all of X⁶, X⁷, and X⁸ are not hydrogen atoms at the same time.

As the heteroaryl group represented by R⁴, a heteroaryl group having 4 to 20 carbon atoms is preferable, a heteroaryl group having 4 to 13 carbon atoms is more preferable, and a heteroaryl group having 4 to 9 carbon atoms is still more preferable.

Examples of the substituent which can be included in the heteroaryl group represented by R⁴ include a nitro group, a halogen atom, an alkyl group having 1 to 3 carbon atoms, a haloalkyl group having 1 to 3 carbon atoms, an acetyl group, a haloacetyl group, and an alkoxy group having 1 to 3 carbon atoms.

Examples of the halogen atom represented by X⁶, X⁷, and X⁸ include a fluorine atom, a chlorine atom, a bromine atom, and an iodine atom, and a chlorine atom, a bromine atom, or an iodine atom is preferable and a chlorine atom or a bromine atom is more preferable.

Examples of the compound represented by General Formula (7) include tribromoquinaldine, 2-tribromomethyl-4-methylquinoline, 4-tribromomethylpyrimidine, 4-phenyl-6-tribromomethylpyrimidine, 2-trichloromethyl-6-nitrobenzothiazole, 1-phenyl-3-trichloromethylpyrazole, 2,5-ditribromomethyl-3,4-dibromothiophene, and 2-trichloromethyl-3-(p-butoxystyryl)-1,3,4-oxadiazole.

In addition, a halomethyl-s-triazine-based compound (compound having a halomethyl-s-triazine structure) is also preferable as the compound represented by General Formula (7), and examples thereof include vinyl-halomethyl-s-triazine compounds described in JP1984-1281B (JP-S59-1281B), and 2-(naphtho-1-yl)-4,6-bis-halomethyl-s-triazine compounds and 4-(p-aminophenyl)-2,6-di-halomethyl-s-triazine compounds described in JP1978-133428A (JP-S53-133428A).

Other examples thereof include 2,4-bis(trichloromethyl)-6-p-methoxystyryl-s-triazine, 2,6-bis(trichloromethyl)-4-(3,4-methylenedioxyphenyl)-1,3,5-triazine, 2,6-bis(trichloromethyl)-4-(4-methoxyphenyl)-1,3,5-triazine, 2,4-bis(trichloromethyl)-6-(1-p-dimethylaminophenyl-1,3-butadienyl)-s-triazine, 2-trichloromethyl-4-amino-6-p-methoxystyryl-s-triazine, 2-(naphtho-1-yl)-4,6-bis-trichloromethyl-s-triazine, 2-(4-methoxy-naphtho-1-yl)-4,6-bis-trichloromethyl-s-triazine, 2-(4-ethoxy-naphtho-1-yl)-4,6-bis-trichloromethyl-s-triazine, 2-(4-butoxy-naphtho-1-yl)-4,6-bis-trichloromethyl-s-triazine, 2-[4-(2-methoxyethyl)-naphtho-1-yl]-4,6-bis-trichloromethyl-s-triazine, 2-[4-(2-ethoxyethyl)-naphtho-1-yl]-4,6-bis-trichloromethyl-s-triazine, 2-[4-(2-butoxyethyl)-naphtho-1-yl]-4,6-bis-trichloromethyl-s-triazine, 2-(2-methoxy-naphtho-1-yl)-4,6-bis-trichloromethyl-s-triazine, 2-(6-methoxy-5-methyl-naphtho-2-yl)-4,6-bis-trichloromethyl-s-triazine, 2-(6-methoxy-naphtho-2-yl)-4,6-bis-trichloromethyl-s-triazine, 2-(5-methoxy-naphtho-1-yl)-4,6-bis-trichloromethyl-s-triazine, 2-(4,7-dimethoxy-naphtho-1-yl)-4,6-bis-trichloromethyl-s-triazine, 2-(6-ethoxy-naphtho-2-yl)-4,6-bis-trichloromethyl-s-triazine, 2-(4,5-dimethoxy-naphtho-1-yl)-4,6-bis-trichloromethyl-s-triazine, 4-[p-N,N-di(ethoxycarbonylmethyl)aminophenyl]-2,6-di(trichloromethyl)-s-triazine, 4-[o-methyl-p-N,N-di(ethoxycarbonylmethyl)aminophenyl]-2,6-di(trichloromethyl)-s-triazine, 4-[p-N,N-di(chloroethyl)aminophenyl]-2,6-di(trichloromethyl)-s-triazine, 4-[o-methyl-p-N,N-di(chloroethyl)aminophenyl]-2,6-di(trichloromethyl)-s-triazine, 4-(p-N-chloroethylaminophenyl)-2,6-di(trichloromethyl)-s-triazine, 4-(p-N-ethoxycarbonylmethylaminophenyl)-2,6-di(trichloromethyl)-s-triazine, 4-[p-N,N-di(phenyl)aminophenyl]-2,6-di(trichloromethyl)-s-triazine, 4-(p-N-chloroethylcarbonylaminophenyl)-2,6-di(trichloromethyl)-s-triazine, 4-[p-N-(p-methoxyphenyl)carbonylaminophenyl]-2,6-di(trichloromethyl)-s-triazine, 4-[m-N,N-di(ethoxycarbonylmethyl)aminophenyl]-2,6-di(trichloromethyl)-s-triazine, 4-[m-bromo-p-N,N-di(ethoxycarbonylmethyl)aminophenyl]-2,6-di(trichloromethyl)-s-triazine, 4-[m-chloro-p-N,N-di(ethoxycarbonylmethyl)aminophenyl]-2,6-di(trichloromethyl)-s-triazine, 4-[m-fluoro-p-N,N-di(ethoxycarbonylmethyl)aminophenyl]-2,6-di(trichloromethyl)-s-triazine, 4-[o-bromo-p-N,N-di(ethoxycarbonylmethyl)aminophenyl]-2,6-di(trichloromethyl)-s-triazine, 4-[o-chloro-p-N,N-di(ethoxycarbonylmethyl)aminophenyl]-2,6-di(trichloromethyl)-s-triazine, 4-[o-fluoro-p-N,N-di(ethoxycarbonylmethyl)aminophenyl]-2,6-di(trichloromethyl)-s-triazine, 4-[o-bromo-p-N,N-di(chloroethyl)aminophenyl]-2,6-di(trichloromethyl)-s-triazine, 4-[o-chloro-p-N,N-di(chloroethyl)aminophenyl]-2,6-di(trichloromethyl)-s-triazine, 4-[o-fluoro-p-N,N-di(chloroethyl)aminophenyl]-2,6-di(trichloromethyl)-s-triazine, 4-[m-bromo-p-N,N-di(chloroethyl)aminophenyl]-2,6-di(trichloromethyl)-s-triazine, 4-[m-chloro-p-N,N-di(chloroethyl)aminophenyl]-2,6-di(trichloromethyl)-s-triazine, 4-[m-fluoro-p-N,N-di(chloroethyl)aminophenyl]-2,6-di(trichloromethyl)-s-triazine, 4-(m-bromo-p-N-ethoxycarbonylmethylaminophenyl)-2,6-di(trichloromethyl)-s-triazine, 4-(m-chloro-p-N-ethoxycarbonylmethylaminophenyl)-2,6-di(trichloromethyl)-s-triazine, 4-(m-fluoro-p-N-ethoxycarbonylmethylaminophenyl)-2,6-di(trichloromethyl)-s-triazine, 4-(o-bromo-p-N-ethoxycarbonylmethylaminophenyl)-2,6-di(trichloromethyl)-s-triazine, 4-(o-chloro-p-N-ethoxycarbonylmethylaminophenyl)-2,6-di(trichloromethyl)-s-triazine, 4-(o-fluoro-p-N-ethoxycarbonylmethylaminophenyl)-2,6-di(trichloromethyl)-s-triazine, 4-(m-bromo-p-N-chloroethylaminophenyl)-2,6-di(trichloromethyl)-s-triazine, 4-(m-chloro-p-N-chloroethylaminophenyl)-2,6-di(trichloromethyl)-s-triazine, 4-(m-fluoro-p-N-chloroethylaminophenyl)-2,6-di(trichloromethyl)-s-triazine, 4-(o-bromo-p-N-chloroethylaminophenyl)-2,6-di(trichloromethyl)-s-triazine, 4-(o-chloro-p-N-chloroethylaminophenyl)-2,6-di(trichloromethyl)-s-triazine, and 4-(o-fluoro-p-N-chloroethylaminophenyl)-2,6-di(trichloromethyl)-s-triazine.

Among these, as the organic halogen compound, the compound represented by General Formula (3), the compound represented by General Formula (6), or the compound represented by General Formula (7) is preferable, and from the viewpoint that the effect of the present invention is more excellent, the compound represented by General Formula (7) is more preferable, and the halomethyl-s-triazine-based compound is particularly preferable. That is, it is particularly preferable that the photoactivator has a halomethyl-s-triazine structure. The reason why the effect of the present invention is more excellent is not clear, but it is presumed that the above-described compound represented by General Formula (7) is likely to accept electrons from the sensitizer in an excited state, and can efficiently generate acid or radical.

The organic halogen compound may be used alone or in combination of two or more kinds thereof.

Photoacid Generator

The photoacid generator is preferably a compound which is cleaved by ultraviolet rays to generate an acid and can cause the forming of the color-forming agent by the action of acid.

Examples of the photoacid generator include a non-ionic photoacid generator and an ionic photoacid generator, and from the viewpoint that the effect of the present invention is more excellent, a non-ionic photoacid generator is preferable. Examples of the non-ionic photoacid generator include an organic halogen compound and an oxime compound, and among these, from the viewpoint that the effect of the present invention is more excellent, an organic halogen compound is preferable, and the compound represented by General Formula (7) described above is more preferable.

From the viewpoint that the gradation properties of the colored part are more excellent, the organic halogen compound is preferably a compound in which the number of halogen atoms in the molecule is 3 or more. The upper limit value of the number of halogen atoms is preferably 9 or less.

The organic halogen compound may be used alone or in combination of two or more kinds thereof.

Specific examples of the organic halogen compound include the same organic halogen compounds as those mentioned as the photooxidant in the upper part.

Examples of the ionic photoacid generator include a diazonium salt, an iodonium salt, and a sulfonium salt, and an iodonium salt or a sulfonium salt is preferable. Examples of the ionic photoacid color-forming agent include compounds described in JP1987-161860A (JP-S62-161860A), JP1986-067034A (JP-S61-067034A), and JP1987-050382A (JP-S62-050382A), the contents of which are incorporated in the present specification.

In addition, the photoacid generator is not particularly limited as long as it is a compound which generates an acid by light, and the photoacid generator may be a photoacid generator which generates an inorganic acid such as a hydrogen halide (for example, hydrochloric acid), a sulfuric acid, and a nitric acid, or may be a photoacid generator which generates an organic acid such as a carboxylic acid and a sulfonic acid. From the viewpoint that the effect of the present invention is more excellent, a photoacid generator which generates an inorganic acid is preferable, and a photoacid generator which generates a hydrogen halide is more preferable.

Specific examples of the photoacid generator include triarylsulfonium hexafluorophosphate, triarylsulfonium arsenate and triarylsulfonium antimonate, diaryliodonium hexafluorophosphate, diaryliodonium arsenate and diaryliodonium antimonate, dialkylphenacylsulfonium tetrafluoroborate and dialkylphenacylsulfonium hexafluorophosphate, dialkyl-4-hydroxyphenylsulfonium tetrafluoroborate and dialkyl-4-hydroxyphenylsulfonium hexafluorophosphate, N-bromosuccinimide, tribromomethylphenylsulfone, diphenyliodine, and 2-trichloromethyl-5-(p-butoxystyryl)-1,3,4-oxadiazole.

In addition, the above-described halomethyl-s-triazine-based compound is also particularly preferable as the photooxidant of the present invention.

(Sensitizer)

The ultraviolet-sensing member according to the first embodiment of the present invention contains a sensitizer having an absorption maximal wavelength in a range of 200 to 230 nm.

The sensitizer of the present invention has a function of absorbing light having a wavelength of 200 to 230 nm to be excited, and then applying electrons, holes, or energy from the excited state to the photoactivator, thereby generating a radical or an acid.

In addition, from the viewpoint of detecting the irradiation amount of light having a wavelength of 222 nm, which inactivates the novel coronavirus infection, with high sensitivity, the sensitizer of the present invention has an absorption maximal wavelength in a wavelength range of 200 to 230 nm.

In a case where the sensitizer of the present invention is a compound which generates a radical or an acid with the photoactivator from the excited state by donating electrons to the photoactivator (hereinafter, referred to as an electron-donating sensitizer), it is preferable that a change in free energy represented by the following expression (III) is negative from the viewpoint of efficient electron migration from the sensitizer to the photoactivator.

ΔG=F×E_{D}−F×E_{A}−E*_{D}−N_{A}×e²/(4π×ε×ε₀×r) Expression (III)

Here,
ΔG represents a change in free energy of the reaction,
F represents a Faraday constant,
E_{D} represents an oxidation potential of the sensitizer,
E_{A} represents a reduction potential of the photoinitiator,
E*_{D} represents an excitation energy of the sensitizer,
N_{A} represents an Avogadro constant,
e represents a charge of electron,
ε represents a dielectric constant of the binder resin,
ε₀ represents a dielectric constant of vacuum, and
r represents a distance between two charges.

Various compounds can be used as the sensitizer of the present invention as long as they absorb the light having a wavelength of 200 to 230 nm, but from the viewpoint that ΔG is negative, a condensed ring aromatic compound is preferable, and naphthalenes, anthracenes, pyrenes, carbazoles, dibenzofurans, dibenzothiophenes, or the like can be preferably used. Among these, naphthalenes are preferable from the viewpoint that they have a large light absorption coefficient at a wavelength of 200 to 230 nm. That is, it is preferable that the sensitizer has a naphthalene structure.

The sensitizer may be used alone or in combination of two or more kinds thereof.

Specific examples of the compound which preferably functions as the sensitizer of the present invention will be described below, but the present invention is not limited thereto.

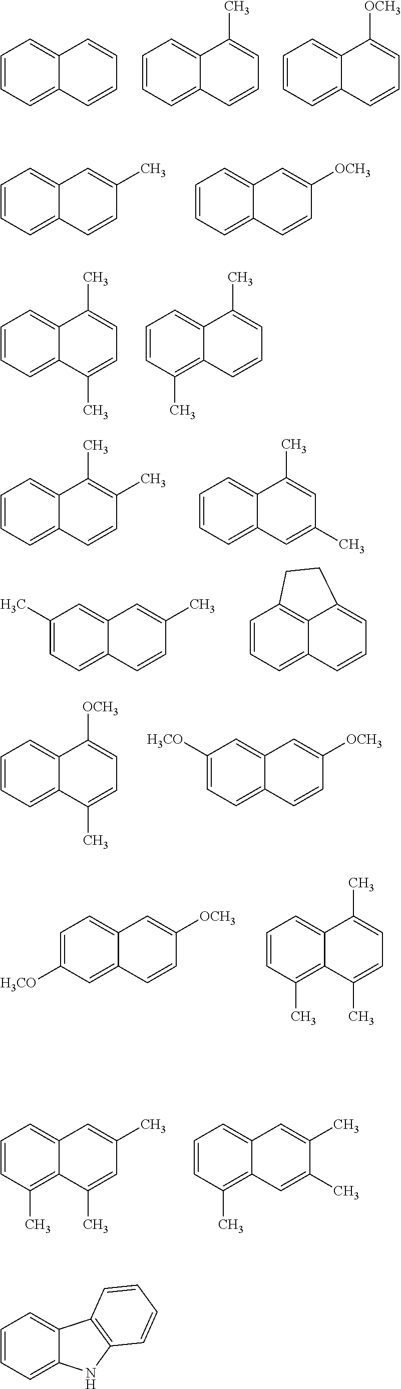

An addition amount of the sensitizer of the present invention is preferably 0.01 parts by mass or more and 50 parts by mass or less, and more preferably 0.1 parts by mass or more and 20 parts by mass or less with respect to 100 parts by mass of the ultraviolet-sensing layer.

In addition, a molar ratio of the sensitizer of the present invention to the photooxidant of the present invention is preferably 0.1 or more and 50 or less, and more preferably 1.0 or more and 30 or less.

Furthermore, from the viewpoint that the sensitizer of the present invention can efficiently absorb light having a wavelength of 200 to 230 nm, a molar ratio of the sensitizer of the present invention to the color-forming agent of the present invention is preferably 0.1 or more, and more preferably 1 or more. The upper limit thereof is not particularly limited, but is practically 50 or less, and is preferably 30 or less, more preferably 10 or less, and still more preferably 5 or less.

Hereinafter, the ultraviolet-sensing member will be described in detail with reference to specific aspects.

An example of the ultraviolet-sensing member according to the first embodiment of the present invention is an ultraviolet-sensing member including an ultraviolet-sensing layer which contains a sensitizer having an absorption maximal wavelength in a range of 200 to 230 nm, a photoactivator, a color-forming agent, and a binder resin.

With the above-described configuration, any one of a difference between C1 and C2, a difference between Y1 and Y2, or a difference between M1 and M2 in the second embodiment described later can be easily controlled to 0.20 or more.

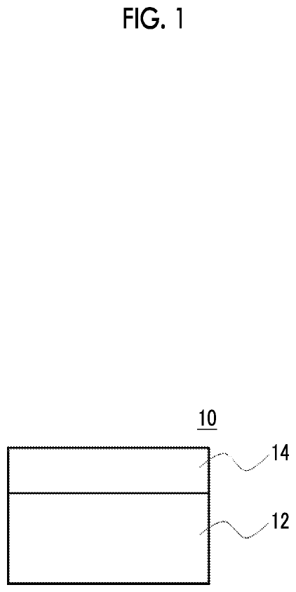

FIG. 1 is a schematic cross-sectional view of an example of the first embodiment of the ultraviolet-sensing member.

An ultraviolet-sensing member 10 includes a support 12, and an ultraviolet-sensing layer 14 which is disposed on one surface of the support 12 and contains a sensitizer having an absorption maximal wavelength in a range of 200 to 230 nm, a photoactivator, a color-forming agent, and a binder resin. In the ultraviolet-sensing layer 14 which is irradiated with ultraviolet rays, a colored part (not shown) which forms color with a color optical density corresponding to an amount of ultraviolet irradiation is formed.

As described above, although FIG. 1 shows an aspect in which the ultraviolet-sensing member is a sheet-like shape, the present invention is not limited to this aspect.

As will be described later, it is sufficient that the ultraviolet-sensing member 10 includes the ultraviolet-sensing layer 14, and the support 12 may not be included.

Furthermore, the ultraviolet-sensing member 10 shown in FIG. 1 has a two-layer configuration of the support 12 and the ultraviolet-sensing layer 14, but the present invention is not limited to this aspect. As will be described later, the ultraviolet-sensing member 10 may include a layer other than the support 12 and the ultraviolet-sensing layer 14 (for example, a reflective layer, a glossy layer, a filter layer, and the like).

In a case where the ultraviolet-sensing layer included in the ultraviolet-sensing member is irradiated with ultraviolet rays for measuring an amount of ultraviolet irradiation, in a region irradiated with ultraviolet rays (ultraviolet-irradiated region), a colored part (color-formed image) is formed with a color optical density corresponding to the amount of ultraviolet irradiation (for example, integrated illuminance). The fact that color is formed with the color optical density corresponding to the amount of ultraviolet irradiation means that the color-formed image has gradation properties according to the amount of ultraviolet irradiation.

In a case where the ultraviolet-sensing layer is irradiated with ultraviolet rays, a color-forming agent present in the ultraviolet-irradiated region usually forms color.

Specifically, the sensitizer having an absorption maximal wavelength in a range of 200 to 230 nm or the photoactivator absorbs ultraviolet rays and is activated to generate an acid and/or a radical, and the color-forming agent forms color by reaction with this acid and/or radical. In this case, an amount of acid and/or radical generated from the photoactivator varies depending on the irradiated amount of ultraviolet irradiation, and an amount of the color-forming agent which forms color also varies depending on the amount of acid and/or radical generated from the photoactivator. As a result, in the ultraviolet-irradiated region of the ultraviolet-sensing layer, shade of the color optical density is generated according to the irradiated amount of ultraviolet irradiation, and the colored part is formed with the color optical density corresponding to the amount of ultraviolet irradiation.

A lower limit value of a thickness of the ultraviolet-sensing member 10 is preferably 0.1 m or more and more preferably 0.5 m or more. In addition, the upper limit value thereof is preferably 1 cm or less and more preferably 2 mm or less.

Hereinafter, each member according to an example of the first embodiment of the ultraviolet-sensing member will be described in detail.

<<Support>>

The support is a member for supporting the ultraviolet-sensing layer.

In a case where the ultraviolet-sensing layer itself can be handled, the ultraviolet-sensing member may not include the support.

Examples of the support include a resin sheet, paper (including synthetic paper), cloth (including woven fabric and nonwoven fabric), glass, wood, and metal. As the support, a resin sheet or paper is preferable, a resin sheet or synthetic paper is more preferable, and a resin sheet is still more preferable.

Examples of a material of the resin sheet include a polyethylene-based resin, a polypropylene-based resin, a cyclic polyolefin-based resin, a polystyrene-based resin, an acrylonitrile-styrene copolymer, an acrylonitrile-butadiene-styrene copolymer, a polyvinyl chloride-based resin, a fluorine-based resin, a poly(meth)acrylic resin, a polycarbonate-based resin, a polyester-based resin (polyethylene terephthalate, polyethylene naphthalate, and the like), a polyamide-based resin such as various nylons, a polyimide-based resin, a polyamide-imide-based resin, a polyaryl phthalate-based resin, a silicone-based resin, a polysulfone-based resin, a polyphenylene sulfide-based resin, a polyethersulfone-based resin, a polyurethane-based resin, an acetal-based resin, and a cellulose-based resin.

Examples of the synthetic paper include paper in which many microvoids are formed by biaxially stretching polypropylene or polyethylene terephthalate (YUPO and the like); paper produced from synthetic fibers such as polyethylene, polypropylene, polyethylene terephthalate, and polyamide; and paper in which these papers are laminated on part, one side, or both sides thereof.

In addition, examples of another suitable aspect of the resin sheet include a white resin sheet formed by dispersing a white pigment in a resin. Examples of a material of the resin in the above-described white resin sheet include the same materials as those in the resin sheet described above.

The white resin sheet has ultraviolet reflectivity. Therefore, in a case where the support is the white resin sheet, since ultraviolet rays irradiated to the ultraviolet-sensing member are reflected by the support, it is possible to suppress scattering of the ultraviolet rays inside the ultraviolet-sensing member. As a result, accuracy of detecting the amount of ultraviolet irradiation in the ultraviolet-sensing member can be further improved.

As the white pigment, white pigments described in paragraph 0080 of WO2016/017701A can be referred to, the contents of which are incorporated in the present specification.

As the white resin sheet, for example, a white polyester sheet is preferable, and a white polyethylene terephthalate sheet is more preferable.

Examples of a commercially available product of the white resin sheet include YUPO (manufactured by YUPO Corporation), LUMIRROR (manufactured by Toray Industries Inc.), and CRISPER (manufactured by Toyobo Co., Ltd.).

A lower limit value of a thickness of the support is preferably 5 μm or more, more preferably 25 μm or more, and still more preferably 50 μm or more. In addition, the upper limit value thereof is preferably 1 cm or less, more preferably 2 mm or less, and still more preferably 500 m.

<<Ultraviolet-Sensing Layer>>

The ultraviolet-sensing layer contains a sensitizer having an absorption maximal wavelength in a range of 200 to 230 nm, a photoactivator, a color-forming agent, and a binder resin.

Hereinafter, various components which can be contained in the ultraviolet-sensing layer will be described in detail.

Aspects of the sensitizer, the photoactivator, and the color-forming agent are as described above.

<Binder Resin>

The ultraviolet-sensing layer contains a binder resin.

Various resins can be used as the binder resin of the present invention, but from the viewpoint of high transmittance of ultraviolet rays having a wavelength of 200 to 230 nm, an aliphatic resin is preferable, and a polyvinyl acetal resin, a polyvinyl butyral resin, a cellulose ester resin, an acrylic resin, a polycycloolefin resin, an ethylene/vinyl acetate copolymer resin, a polyamide resin, a polyurethane resin, a polyethylene resin, a polypropylene resin, or a polyester resin is preferable. Among these, a polyvinyl acetal resin, a polyvinyl butyral resin, or a cellulose ether resin is particularly preferable from the viewpoint of color forming of the color-forming agent.

Preferred examples of the polyvinyl acetal resin and the polyvinyl butyral resin of the present invention include S LEC (manufactured by Sekisui Chemical Co., Ltd.), Mowital (manufactured by KURARAY CO., LTD.), and VINYLEC (manufactured by JNC Corporation), in which various grades are available on the market.

Specific examples thereof include Poval PVA 403, PVA-624, PVA-706, PVA 102, PVA 203, PVA 505, PVA HC, L-8, PVA-CST, L-9-78, LL-02, C-506, KL-05, and KL-506; S LEC BL-1, S LEC BL-1H, S LEC BL-2, S LEC BL-2H, S LEC BL-5, S LEC BL-10, S LEC BL-S, S LEC BX-L, S LEC BM-1, S LEC BM-S, S LEC BH-3, S LEC BX-1, S LEC KS-1, S LEC KS-10, and S LEC KS-3; Mowital B16H, Mowital B60HH, Mowital 30T, Mowital 30HH, and Mowital 60T; and VINYLEC K, VINYLEC L, VINYLEC H, and VINYLEC E, but the present invention is not limited thereto.

Preferred examples of the cellulose ether resin of the present invention include methylcellulose, ethylcellulose, hydroxypropylmethylcellulose, and hydroxyethylmethylcellulose, and for example, METOLOSE SM series, METOLOSE SH series, and METOLOSE SE series 90SH-100 manufactured by Shin-Etsu Chemical Co., Ltd., Marpolose series manufactured by Matsumoto Yushi-Seiyaku Co., Ltd., and Ethocel series manufactured by Dow Chemical Company can be obtained from the market.

The binder resin may be used alone or in combination of two or more kinds thereof.

With respect to the total mass of the ultraviolet-sensing layer, the ultraviolet-sensing layer of the present invention preferably contains 5% by mass or more of the above-described resin, more preferably contains 20% by mass or more of the above-described resin, and still more preferably contains 50% by mass or more of the above-described resin. The upper limit thereof is not particularly limited, and examples thereof include 90% by mass or less.

A mass (coating amount of solid content) per unit area of the ultraviolet-sensing layer is not particularly limited, but for example, is preferably 0.1 to 30 g/m², more preferably 0.5 to 25 g/m², and still more preferably 1 to 10 g/m².

A thickness of the ultraviolet-sensing layer is preferably 0.1 to 30 μm, more preferably 0.5 to 25 μm, and still more preferably 1 to 10 μm.

<Method for Forming Ultraviolet-Sensing Layer>

A method for forming the above-described ultraviolet-sensing layer is not particularly limited, and examples thereof include known methods.

Examples thereof include a method of forming the ultraviolet-sensing layer by applying a liquid for forming an ultraviolet-sensing layer.

The method of applying the liquid for forming an ultraviolet-sensing layer is not particularly limited, examples of a coating machine used for the applying include an air knife coater, a rod coater, a bar coater, a curtain coater, a gravure coater, an extrusion coater, a die coater, a slide bead coater, and a blade coater.

After the liquid for forming an ultraviolet-sensing layer is applied onto the support, the coating film may be subjected to a drying treatment, as necessary. Examples of the drying treatment include a heating treatment.

Although the method for forming the ultraviolet-sensing layer on the support has been described, the present invention is not limited to the above-described aspect. For example, after forming the ultraviolet-sensing layer on a temporary support, the temporary support may be peeled off to form the ultraviolet-sensing member including the ultraviolet-sensing layer.

The temporary support is not particularly limited as long as it is a peelable support.

<<Other Layers>>

The ultraviolet-sensing member may include a layer other than the support and the ultraviolet-sensing layer described above.

Examples of other layers include a reflective layer, a glossy layer, a filter layer, and a sensitivity-adjusting layer.

<Reflective Layer>

The ultraviolet-sensing member may further include a reflective layer.

In a case where the ultraviolet-sensing layer includes a reflective layer, since ultraviolet rays irradiated to the ultraviolet-sensing member can be reflected by the layer having ultraviolet reflectivity, scattering of the ultraviolet rays inside the ultraviolet-sensing member can be suppressed, and detection accuracy of the amount of ultraviolet irradiation can be further improved.

A reflectivity of the reflective layer with respect to light having a wavelength of 200 to 380 nm is preferably 10% or more, and more preferably 50% or more. The reflectivity can be measured, for example, by diffusion reflection measurement using an ultraviolet-visible spectrophotometer (UV-2700, Shimadzu Corporation).

In a case where the support is disposed adjacent to the reflective layer, an adhesive layer may be provided between the support and the reflective layer.

As the reflective layer, the adhesive layer, and manufacturing methods thereof, the reflective layer, the adhesive layer, and manufacturing methods thereof, which are described in paragraphs 0082 to 0091 of WO2016/017701A, can be referred to. The contents thereof are incorporated in the present specification.

<Glossy Layer>

The ultraviolet-sensing member may further include a glossy layer.

In a case where the ultraviolet-sensing layer includes a glossy layer, visibility of front and back surfaces can be improved.

As the glossy layer and a method for producing the glossy layer, glossy layers and method for producing the glossy layer, described in paragraphs 0092 to 0094 of WO2016/017701A, can be referred to, the contents of which are incorporated in the present specification.

<Filter Layer>

It is preferable that the ultraviolet-sensing member further includes a filter layer.

The filter layer is a layer which selectively transmits light having a specific wavelength. Here, the “selectively transmits light having a specific wavelength” means transmitting the light having a specific wavelength and shielding other lights. For example, a transmittance of light having a wavelength to be transmitted is preferably 70% or more, more preferably 80% or more, and still more preferably 90% or more. A transmittance of light having a wavelength to be shielded is preferably 30% or less, more preferably 20% or less, and still more preferably 10% or less.

The filter layer is preferably a filter layer which shields light having a wavelength of 300 nm or more, and more preferably a filter layer which shields light having a wavelength of more than 230 nm. An ultraviolet band pass filter, a filter containing a dielectric, or the like is preferably used.

Spectral characteristics of the filter layer and the sensitivity-adjusting layer described later can be measured using, for example, an ultraviolet-visible spectrophotometer (UV-2700, Shimadzu Corporation).

The filter layer preferably has an ultraviolet absorber from the viewpoint of shielding light having a wavelength other than the specific wavelength. As the ultraviolet absorber, a known ultraviolet absorber can be used.

As the filter layer and a method for producing the filter layer, filter layers and method for producing the filter layer, described in paragraphs 0016 to 0026 of WO2016/017701A, can be referred to, the contents of which are incorporated in the present specification.

<Sensitivity-Adjusting Layer>

In a case where the ultraviolet-sensing member includes the filter layer, a sensitivity-adjusting layer may be further provided on a surface of the filter layer.

As the sensitivity-adjusting layer and a method for producing the glossy layer, sensitivity-adjusting layers and method for producing the sensitivity-adjusting layer, described in paragraphs 0095 to 0109 of WO2016/017701A, can be referred to, the contents of which are incorporated in the present specification.

### Second Embodiment

The ultraviolet-sensing member according to the second embodiment of the present invention is a ultraviolet-sensing member in which, in a case where, using a KrCl (krypton chloride) excimer lamp as a light source, the ultraviolet-sensing member is irradiated with light through a filter which substantially shields light having a wavelength of 230 to 300 nm until an irradiation amount of light having a wavelength of 222 nm reaches 3 mJ/cm², using a spectrocolorimeter X-Rite (manufactured by X-Rite), values of cyan optical densities, magenta optical densities, and yellow optical densities are measured with the ultraviolet-sensing member before the light irradiation and the ultraviolet-sensing member after the light irradiation, and a value of a cyan optical density of the ultraviolet-sensing member before the light irradiation is denoted as C1, a value of a cyan optical density of the ultraviolet-sensing member after the light irradiation is denoted as C2, a value of a yellow optical density of the ultraviolet-sensing member before the light irradiation is denoted as Y1, a value of a yellow optical density of the ultraviolet-sensing member after the light irradiation is denoted as Y2, a value of a magenta optical density of the ultraviolet-sensing member before the light irradiation is denoted as M1, and a value of a magenta optical density of the ultraviolet-sensing member after the light irradiation is denoted as M2, any one of a difference between C1 and C2, a difference between Y1 and Y2, or a difference between M1 and M2 is 0.20 or more, and a rate of change in optical density of a color which exhibits the largest difference among the difference between C1 and C2, the difference between Y1 and Y2, and the difference between M1 and M2, represented by Expression (II) described later, is −50% or more and 50% or less.

In the second embodiment of the ultraviolet-sensing member according to the present invention, in a case of being irradiated with ultraviolet rays with a wavelength of 222 nm at an integrated illuminance (irradiation amount) of 3 mJ/cm², there is a change in tint, so that it is easy to determine whether the irradiation amount which inactivates the novel coronavirus has been irradiated.

In addition, in the second embodiment of the ultraviolet-sensing member according to the present invention, it is further found that a change in optical density in a case of being stored at room temperature after the light irradiation is small.

In the following, first, the above-described characteristics of the ultraviolet-sensing member will be described in detail.

First, using a KrCl excimer lamp as a light source, the ultraviolet-sensing member is irradiated with light through a filter which substantially shields light having a wavelength of 230 to 300 nm until an irradiation amount of light having a wavelength of 222 nm reaches 3 mJ/cm². From the viewpoint of easy handling, a size of the ultraviolet-sensing member to be irradiated with light is preferably a size of a length of 5 mm to 10 m in a vertical direction and a length of 5 mm to 300 mm in a horizontal direction.

The filter which substantially shields light having a wavelength of 230 to 300 nm means a filter which shields 70% to 100% of the light having a wavelength of 230 to 300 nm.

In other words, the above-described filter is a filter having a maximum transmittance of 30% or less in the wavelength range of 230 to 300 nm. As such a filter, a chemical filter or a filter containing a dielectric is usually used.

As an ultraviolet irradiation device in which the KrCl excimer lamp is used as the light source and the filter which substantially shields light having a wavelength of 230 to 300 nm is provided, an ultraviolet irradiation device Care 222 (registered trademark) available from Ushio Inc. may be used. In the Care 222 (registered trademark), a lamp in which a main wavelength is ultraviolet rays with a wavelength of 222 nm, which is suitable for sterilization, is combined with a filter that limits light to a wavelength range (wavelength of 200 to 230 nm) which is harmless to the human body. Therefore, in a case where light irradiation is performed using the Care 222 (registered trademark), light having a wavelength of 222 nm is mainly irradiated.

Illuminance and irradiation time in the case of the irradiation are not particularly limited, and the irradiation amount can be set to 3 mJ/cm² by adjusting a distance between the light source and the ultraviolet-sensing member and the irradiation time. As an example, the irradiation amount is preferably 3 mJ/cm² in approximately 10 seconds (preferably, 9 seconds).

In addition, using a known ultraviolet-measuring device (for example, Handheld Light Meter UIT2400 (manufactured by Ushio Inc.), it may be measured whether or not a predetermined irradiation amount has been applied to the ultraviolet-sensing member.

Next, using a spectrocolorimeter X-Rite (manufactured by X-Rite), values of cyan optical densities, magenta optical densities, and yellow optical densities are measured with the ultraviolet-sensing member before the light irradiation and the ultraviolet-sensing member after the light irradiation.

In a case where the ultraviolet-sensing member before the light irradiation and the ultraviolet-sensing member after the light irradiation are to be measured, by measuring optical densities (OD) in each of Cyan (C) mode, Magenta (M) mode, and Yellow (Y) mode, a value (C value) of the cyan optical density, a value (M value) of the magenta optical density, and a value (Y value) of the yellow optical density are measured respectively.

Next, in a case where a value of a cyan optical density of the ultraviolet-sensing member before the light irradiation is denoted as C1, a value of a cyan optical density of the ultraviolet-sensing member after 2 hours from the light irradiation is denoted as C2, a value of a yellow optical density of the ultraviolet-sensing member before the light irradiation is denoted as Y1, a value of a yellow optical density of the ultraviolet-sensing member after 2 hours from the light irradiation is denoted as Y2, a value of a magenta optical density of the ultraviolet-sensing member before the light irradiation is denoted as M1, and a value of a magenta optical density of the ultraviolet-sensing member after 2 hours from the light irradiation is denoted as M2, a difference between C1 and C2, a difference between Y1 and Y2, and a difference between M1 and M2 are calculated.

The difference between C1 and C2 is a value obtained by subtracting the smaller one of C1 and C2 from the larger one thereof. In a case where the values of C1 and C2 are the same, the difference between the two is 0.

The difference between Y1 and Y2 is a value obtained by subtracting the smaller one of Y1 and Y2 from the larger one thereof. In a case where the values of Y1 and Y2 are the same, the difference between the two is 0.

The difference between M1 and M2 is a value obtained by subtracting the smaller one of M1 and M2 from the larger one thereof. In a case where the values of M1 and M2 are the same, the difference between the two is 0.

Any one of the difference between C1 and C2, the difference between Y1 and Y2, or the difference between M1 and M2, which are obtained by the above-described procedure, is 0.20 or more, preferably 0.2 to 1.5 and more preferably 0.26 to 1.1.

Among these, from the viewpoint that the effect of the present invention is more excellent, it is preferable that any one of the difference between C1 and C2 or the difference between M1 and M2 is 0.20 or more.

The difference between C1 and C2 is preferably 0.20 or more, more preferably 0.20 to 1.50, and still more preferably 0.26 to 1.10.

The value of C1 is often 0.00 to 0.10, preferably 0.00 to 0.05.

The value of C2 is preferably 0.20 or more, and more preferably 0.20 to 1.50.

The difference between M1 and M2 is preferably 0.20 or more, more preferably 0.20 to 1.50, and still more preferably 0.26 to 1.10.

The value of M1 is often 0.00 to 0.1, preferably 0.00 to 0.05.

The value of M2 is preferably 0.20 or more, and more preferably 0.20 to 1.50.

The difference between Y1 and Y2 is preferably 0.20 or more, more preferably 0.20 to 1.50, and still more preferably 0.26 to 1.10.

The value of Y1 is often 0.00 to 0.10, and more preferably 0.00 to 0.05.

The value of Y2 is preferably 0.20 or more, and more preferably 0.20 to 1.50.

Next, in a case where a value of a cyan optical density of the ultraviolet-sensing member after 24 hours from the light irradiation is denoted as C3, a value of a yellow optical density of the ultraviolet-sensing member after 24 hours from the light irradiation is denoted as Y3, and a value of a magenta optical density of the ultraviolet-sensing member after 24 hours from the light irradiation is denoted as M3, from C2 and C3, Y2 and Y3, and M2 and M3, rates of change in yellow optical density, magenta optical density, and cyan optical density are calculated by the following expression (II), respectively.

Rate of change in optical density (%) [(Optical density after 24 hours of irradiation)−(Optical density after 2 hours of irradiation)]//[(Optical density after 2 hours of irradiation)−(Optical density before irradiation)]×100 Expression (II)

In the second embodiment of the ultraviolet-sensing member according to the present invention, the above-described rate of change in optical density of a color which exhibits the largest difference among the difference between C1 and C2, the difference between Y1 and Y2, and the difference between M1 and M2 is −50% or more and 50% or less.

The rates of change in yellow optical density, magenta optical density, and cyan optical density are all preferably −50% or more and 50% or less, more preferably −40% or more and 40% or less, and particularly preferably −30% or more and 30% or less. In a case where the rate of change in optical density satisfies the above-described range, it is possible to stably store evidence for determining whether an irradiation amount which inactivates the novel coronavirus has been irradiated.

In the second embodiment of the ultraviolet-sensing member according to the present invention, the configuration is not particularly limited as long as it satisfies the above-described characteristics, but it is preferable that the ultraviolet-sensing member according to the second embodiment of the present invention contains the materials contained in the first embodiment described above. That is, it is preferable that the ultraviolet-sensing member according to the second embodiment of the present invention contains the sensitizer having an absorption maximal wavelength in a range of 200 to 230 nm, the photoactivator, and the color-forming agent.

In the first embodiment of the ultraviolet-sensing member according to the present invention, with the measurement by the method described in the second embodiment, it is preferable that any one of the difference between C1 and C2, the difference between Y1 and Y2, or the difference between M1 and M2 is 0.20 or more, and the rate of change in optical density of a color which exhibits the largest difference among the difference between C1 and C2, the difference between Y1 and Y2, and the difference between M1 and M2, represented by the above-described expression (II), is −50% or more and 50% or less.

A form of the ultraviolet-sensing member (the first embodiment and the second embodiment) is not particularly limited, and may be a sheet-like shape, and various shapes such as a block shape, for example, a rectangular parallelepiped shape, a cylindrical shape, and the like can be used. Among these, a sheet-like ultraviolet-sensing member, that is, an ultraviolet-sensing sheet is suitably used.

In addition, as the shape of the sheet-like ultraviolet-sensing member, various shapes such as a square shape, a rectangular shape, a circular shape, an elliptical shape, a polygonal shape other than a quadrangular shape, for example, a hexagonal shape and the like, and an amorphous shape can be used. In addition, the sheet-like ultraviolet-sensing member may have a long shape.

The ultraviolet-sensing member may be applied on another member. In a case of being applied on another member, the ultraviolet-sensing member may be attached through an adhesive layer such as a pressure sensitive adhesive and an adhesive, or may be manufactured as a part of another member. Another member is not particularly limited, and examples thereof include a business card, a name tag, a mask, a cloth product (for example, a shirt), a case (for example, a smartphone case), and a paper product (for example, a notebook, a calender, and the like).

[Ultraviolet-Sensing Kit]

In addition, the present invention also relates to an ultraviolet-sensing kit including the above-described ultraviolet-sensing member (the first embodiment and the second embodiment).

The ultraviolet-sensing kit includes at least the above-described ultraviolet-sensing member.

A specific configuration of the ultraviolet-sensing kit is not particularly limited, and examples thereof include an aspect of including the ultraviolet-sensing member and other elements selected from the group consisting of a member having a filter layer which selectively transmits light having a specific wavelength (preferably a filter sheet which shields light having a wavelength of 300 nm or more, and more preferably a filter sheet which shields light having a wavelength of more than 230 nm), a light shielding bag (ultraviolet cut bag), a sample judgment, a limit sample (calibration sheet), a condensing jig such as a lens and a concave mirror, and a holding member which holds the ultraviolet-sensing member.

The above-described holding member may have an opening portion for irradiating the held ultraviolet-sensing member with ultraviolet rays, or the holding member and a determination sample may be integrated.

With regard to the ultraviolet-sensing member included in the ultraviolet-sensing kit according to the embodiment of the present invention, it is preferable that, in a case where the ultraviolet-sensing member is irradiated with light until an irradiation amount of light having a wavelength of 313 nm reaches 9 mJ/cm², using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and a value of a cyan optical density of the ultraviolet-sensing member before the light irradiation is denoted as C4, a value of a cyan optical density of the ultraviolet-sensing member after 2 hours from the light irradiation is denoted as C5, a value of a yellow optical density of the ultraviolet-sensing member before the light irradiation is denoted as Y4, a value of a yellow optical density of the ultraviolet-sensing member after 2 hours of light irradiation is denoted as Y5, a value of a magenta optical density of the ultraviolet-sensing member before the light irradiation is denoted as M4, and a value of a magenta optical density of the ultraviolet-sensing member after 2 hours from the light irradiation is denoted as M5, all of a difference between C4 and C5, a difference between Y4 and Y5, and a difference between M4 and M5 are 0.15 or less.

### EXAMPLES

Hereinafter, the features of the present invention will be more specifically described using Examples and Comparative Examples. The materials, the amounts of materials used, the proportions, the treatment details, the treatment procedure, and the like shown in Examples below may be modified as appropriate as long as the modifications do not depart from the spirit of the present invention. Accordingly, the scope of the present invention should not be construed as being limited by the specific examples given below.

“Part” and “%” that represent compositions in the following Examples are based on the mass unless particularly otherwise described.

All of the steps from the preparation of the liquid for forming an ultraviolet-sensing layer to the production of the ultraviolet-sensing member using the liquid for forming an ultraviolet-sensing layer and the use in the ultraviolet irradiation test were carried out under a yellow lamp so as not to be irradiated with ultraviolet rays.

[Production and Evaluation of Ultraviolet-Sensing Member]

[Production of Ultraviolet-Sensing Layer]

Materials used for producing the ultraviolet-sensing layer are shown below.

<Binder Resin>

(Resin 1)

Polybutyral resin (manufactured by Sekisui Chemical Co., Ltd., S LEC KS-10 (trade name))

(Sensitizer)

Sensitizer 1: 1-methylnaphthalene, manufactured by Tokyo Chemical Industry Co., Ltd.; absorption maximal wavelength in acetonitrile solution=222 nm

Sensitizer 2: 1-methoxynaphthalene, manufactured by Tokyo Chemical Industry Co., Ltd.; absorption maximal wavelength in acetonitrile solution=211 nm

Sensitizer 3: naphthalene, manufactured by Tokyo Chemical Industry Co., Ltd.; absorption maximal wavelength in acetonitrile solution=217 nm

(Photoactivator)

Photoactivator 1: 2-methyl-4,6-bis(trichloromethyl)-1,3,5-triazine, manufactured by Tokyo Chemical Industry Co., Ltd.

(Color-Forming Agent)

Color-forming agent 1: Rhodamine B base, manufactured by Sigma-Aldrich Co. LLC.

(Leveling Agent)

A polymer surfactant composed of the following constitutional components was used as a leveling agent 1. In the following structural formula, a proportion of each constitutional component is a molar ratio, and t-Bu means a tert-butyl group.

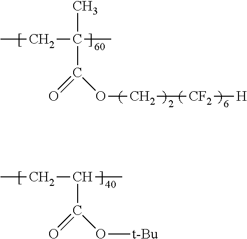

(Base Material 1)

A white polyethylene terephthalate sheet (trade name “CRISPER K1212”, manufactured by Toyobo Co., Ltd.) was used as a base material 1.

### Example 101

<Production of Ultraviolet-Sensing Sheet No. 101>

(1) Preparation of resin solution (liquid for forming ultraviolet-sensing layer)

A liquid (composition) Ba-1 for forming an ultraviolet-sensing layer was prepared by mixing each component with the composition shown below.

Composition of liquid Ba-1 for forming ultraviolet-sensing layer

| | | |
|---|---|---|
| Resin 1 | 93.2 | parts by mass |
| Sensitizer 1 | 1.18 | parts by mass |
| Photoactivator 1 | 4.54 | parts by mass |
| Color-forming agent 1 | 1.10 | parts by mass |
| Leveling agent 1 | 0.08 | parts by mass |
| Methyl ethyl ketone (solvent) | 176.5 | parts by mass |
| Ethanol (solvent) | 294.1 | parts by mass |

Subsequently, the obtained liquid Ba-1 for forming an ultraviolet-sensing layer was filtered using a filter paper (#63, manufactured by Toyo Roshi Kaisha, Ltd.) having an absolute filtration precision of 10 μm, and then further filtered using a sintered metal filter (trade name: Pall filter PMF, media cord: FH025, manufactured by Pall Corporation) having an absolute filtration precision of 2.5 km.

(2) Production of Ultraviolet-Sensing Sheet

The above-described liquid Ba-1 for forming an ultraviolet-sensing layer after the filtration treatment was applied onto the base material 1 using a bar coater so that a film thickness after drying was 2.5 m, and then dried at 120° C. to produce an ultraviolet-sensing sheet No. 101 of the present invention.

### Examples 102 and 103

<Production of Ultraviolet-Sensing Sheets Nos. 102 and 103>

Ultraviolet-sensing sheets Nos. 102 and 103 of the present invention were produced in the same manner as in the ultraviolet-sensing sheet No. 101, except that the type and blending amount of the sensitizer, the type and blending amount of the photoactivator, and the type and blending amount of the color-forming agent were changed as shown in Table 1.

### Reference Example 1

An ultraviolet-sensing sheet No. 201C of Comparative Example was produced in the same manner as in the ultraviolet-sensing sheet No. 101, except that the type and blending amount of the sensitizer, the type and blending amount of the photoactivator, and the type and blending amount of the color-forming agent were changed as shown in Table 1.

### Comparative Example 1

A commercially available UV label (S type, manufactured by NiGK Corporation) was used.

Table 1 is shown below.

TABLE 1

| | | Color-forming agent | | Photoactivator | | | Sensitizer | | |
|---|---|---|---|---|---|---|---|---|---|
| | | | Blending | | Blending | Molar | | Blending | Molar |
| | No. | Type | amount | Type | amount | ratio | Type | amount | ratio |
| Example 1 | 101 | Color-forming | 1.10 | Photoactivator | 4.54 | 5.6 | Sensitizer | 1.18 | 3.3 |
| | | agent 1 | | 1 | | | 1 | | |
| Example 2 | 102 | Color-forming | 1.10 | Photoactivator | 4.54 | 5.6 | Sensitizer | 1.31 | 3.3 |
| | | agent 1 | | 1 | | | 2 | | |
| Example 3 | 103 | Color-forming | 1.10 | Photoactivator | 4.54 | 5.6 | Sensitizer | 1.06 | 3.3 |
| | | agent 1 | | 1 | | | 3 | | |
| Reference | 201C | Color-forming | 1.10 | Photoactivator | 4.54 | 5.6 | None | — | — |
| Example 1 | | agent 1 | | 1 | | | | | |

(Note to Table)

Each of the blending amounts of the color-forming agents indicates parts by mass with respect to 100 parts by mass of the ultraviolet-sensing layer. The blending amount of the photoactivator indicates parts by mass with respect to 100 parts by mass of the ultraviolet-sensing layer. The blending amount of the sensitizer indicates parts by mass with respect to 100 parts by mass of the ultraviolet-sensing layer.

The molar ratio indicates molar amount of blended photoactivator or sensitizer with respect to 1 mol of the color-forming agent.

In addition, the color-forming agent 1 corresponds to a color-forming agent which develops color by action of acid, and exhibits magenta color by the action of acid.

[Measurement and Evaluation of Optical Density]

(Difference in optical density at wavelength of 222 nm)

Using Care 222 (registered trademark), the ultraviolet-sensing member produced in each example was irradiated with light until an irradiation amount of light having a wavelength of 222 nm reached 3 mJ/cm².

After 2 hours from the light irradiation, using a spectrocolorimeter X-Rite (manufactured by X-Rite), values of yellow optical densities, magenta optical densities, and cyan optical densities were measured with the ultraviolet-sensing member before the light irradiation and the ultraviolet-sensing member after the light irradiation, and in a case where a value of a cyan optical density of the ultraviolet-sensing member before the light irradiation was denoted as C1, a value of a cyan optical density of the ultraviolet-sensing member after 2 hours from the light irradiation was denoted as C2, a value of a yellow optical density of the ultraviolet-sensing member before the light irradiation was denoted as Y1, a value of a yellow optical density of the ultraviolet-sensing member after 2 hours was denoted as Y2, a value of a magenta optical density of the ultraviolet-sensing member before the light irradiation was denoted as M1, and a value of a magenta optical density of the ultraviolet-sensing member after 2 hours was denoted as M2, a difference between C1 and C2, a difference between Y1 and Y2, and a difference between M1 and M2 were measured. Among differences in optical density of the difference between C1 and C2, the difference between Y1 and Y2, and the difference between M1 and M2, the largest value is shown in the column of “Difference in optical density” in Table 2.

Furthermore, with regard to a color (cyan, yellow, or magenta) which exhibited the largest value among the differences in optical density of the difference between C1 and C2, the difference between Y1 and Y2, and the difference between M1 and M2, after 24 hours from the irradiation with ultraviolet rays, using a spectrocolorimeter X-Rite (manufactured by X-Rite), optical densities of the ultraviolet-sensing member after the light irradiation were measured, and the rate of change in optical density calculated by the following expression is shown in the column of “Rate of change in optical density” in Table 2.

Rate of change in optical density (%) [(Optical density after 24 hours of irradiation)−(Optical density after 2 hours of irradiation)]//[(Optical density after 2 hours of irradiation)−(Optical density before irradiation)]×100 Expression (II)

(Change in Tint)

It was evaluated whether or not it could be determined by a change in tint of the ultraviolet-sensing member in a case where the ultraviolet-sensing member produced in each example was irradiated with light by adjusting the distance and time so that the integrated illuminance was 3 mJ/cm².
AA: it could be determined very easily.
A: it could be determined easily.

B: it could not be determined easily.

TABLE 2

| | | | Difference in | | |
|---|---|---|---|---|---|
| | | | optical density | | |
| | | Color-forming | Difference | Rate of change in | |
| | | property | between M1 | optical density | Evaluation |
| | Sheet | M1 | and M2 | M | Determination |
| Example 1 | 101 | 0.10 | 0.27 | −2% | AA |
| Example 2 | 102 | 0.13 | 0.22 | −2% | AA |
| Example 3 | 103 | 0.14 | 0.23 | −2% | AA |
| Reference | 201C | 0.12 | 0.18 | −2% | A |
| Example 1 | | | | | |
| Comparative | UV label | 0.065 | 0.07 | −74% | B |
| Example 1 | | | | | |

As shown in Table 2, it was confirmed that the ultraviolet-sensing member according to the embodiment of the present invention exhibited a desired effect.

From the comparison between Examples, Comparative Example 1, and Reference Example 1, it was confirmed that, in a case where the ultraviolet-sensing member contained the sensitizer, the determination could be easily performed.

Furthermore, from the comparison between Examples 1 to 3 and Comparative Example 1, it was found that, in Examples 1 to 3 of the present invention, the difference between the color optical density after 2 hours from the irradiation and the color optical density after 24 hours from the irradiation was small, which is preferable.

### Example 4

The ultraviolet-sensing member of Example 1 was irradiated with ultraviolet rays using a high-pressure mercury lamp manufactured by Heraeus through a bandpass filter with 313 nm until an irradiation amount of light having a wavelength of 313 nm was 9 mJ/cm². Using a spectrocolorimeter X-Rite (manufactured by X-Rite), values of yellow optical densities, magenta optical densities, and cyan optical densities were measured, and in a case of measuring a value of a cyan optical density of the ultraviolet-sensing member before the light irradiation, denoted as C4, a value of a cyan optical density of the ultraviolet-sensing member after 2 hours from the light irradiation, denoted as C5, a value of a yellow optical density of the ultraviolet-sensing member before the light irradiation, denoted as Y4, a value of a yellow optical density of the ultraviolet-sensing member after 2 hours from the light irradiation, denoted as Y5, a value of a magenta optical density of the ultraviolet-sensing member before the light irradiation, denoted as M4, and a value of a magenta optical density of the ultraviolet-sensing member after 2 hours from the light irradiation, denoted as M5, a difference between C4 and C5, a difference between Y4 and Y5, and a difference between M4 and M5 were all 0.15 or less.

### EXPLANATION OF REFERENCES

10: ultraviolet-sensing member
12: support
14: ultraviolet-sensing layer

What is claimed is:

1. An ultraviolet-sensing member comprising:
a sensitizer having an absorption maximal wavelength in a range of 200 to 230 nm;
a photoactivator; and
a color-forming agent.

2. The ultraviolet-sensing member according to claim 1,
wherein the sensitizer has a naphthalene structure.

3. The ultraviolet-sensing member according to claim 1,
wherein the photoactivator has a halomethyl-s-triazine structure.

4. The ultraviolet-sensing member according to claim 1,
wherein the color-forming agent has a spirolactone structure represented by Formula (I), 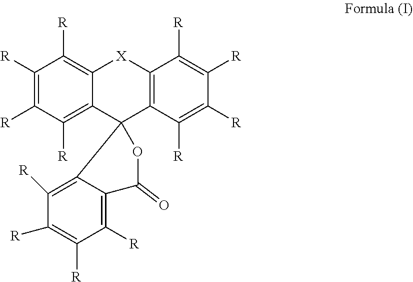
in Formula (I), X represents an oxygen atom, a sulfur atom, or —NR19-, where R19 represents a hydrogen atom, an alkyl group, or an aryl group, and R's each independently represent a hydrogen atom or a monovalent substituent.

5. An ultraviolet-sensing member,
wherein, in a case where, using a KrCl excimer lamp as a light source, the ultraviolet-sensing member is irradiated with light through a filter which substantially shields light having a wavelength of 230 to 300 nm until an irradiation amount of light having a wavelength of 222 nm reaches 3 mJ/cm²,
using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and
a value of a cyan optical density of the ultraviolet-sensing member before the light irradiation is denoted as C1, a value of a cyan optical density of the ultraviolet-sensing member after 2 hours from the light irradiation is denoted as C2, a value of a cyan optical density of the ultraviolet-sensing member after 24 hours from the light irradiation is denoted as C3, a value of a yellow optical density of the ultraviolet-sensing member before the light irradiation is denoted as Y1, a value of a yellow optical density of the ultraviolet-sensing member after 2 hours from the light irradiation is denoted as Y2, a value of a yellow optical density of the ultraviolet-sensing member after 24 hours from the light irradiation is denoted as Y3, a value of a magenta optical density of the ultraviolet-sensing member before the light irradiation is denoted as M1, a value of a magenta optical density of the ultraviolet-sensing member after 2 hours from the light irradiation is denoted as M2, and a value of a magenta optical density of the ultraviolet-sensing member after 24 hours from the light irradiation is denoted as M3,
any one of a difference between C1 and C2, a difference between Y1 and Y2, or a difference between M1 and M2 is 0.20 or more, and
a rate of change in optical density of a color which exhibits a largest difference among the difference between C1 and C2, the difference between Y1 and Y2, and the difference between M1 and M2, represented by the following expression (II), is −50% or more and 50% or less,
rate of change in optical density (%)=[(optical density after 24 hours of irradiation)−(optical density after 2 hours of irradiation)]//[(optical density after 2 hours of irradiation)−(optical density before irradiation)]×100. Expression (II)

6. An ultraviolet-sensing member,
wherein a difference between C1 and C2, a difference between Y1 and Y2, a difference between M1 and M2, and a rate of change in optical density satisfy the requirements of claim 5, and
the ultraviolet-sensing member comprises a sensitizer having an absorption maximal wavelength in a range of 200 to 230 nm, a photoactivator, and a color-forming agent.

7. The ultraviolet-sensing member according to claim 1,
wherein the ultraviolet-sensing member is a sheet-like ultraviolet-sensing member.

8. An ultraviolet-sensing kit comprising:
the ultraviolet-sensing member according to claim 1.

9. An ultraviolet-sensing kit comprising:
the ultraviolet-sensing member according to claim 1,
wherein, in a case where the ultraviolet-sensing member is irradiated with light until an irradiation amount of light having a wavelength of 313 nm reaches 9 mJ/cm²,
using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and
a value of a cyan optical density of the ultraviolet-sensing member before the light irradiation is denoted as C4, a value of a cyan optical density of the ultraviolet-sensing member after 2 hours from the light irradiation is denoted as C5, a value of a yellow optical density of the ultraviolet-sensing member before the light irradiation is denoted as Y4, a value of a yellow optical density of the ultraviolet-sensing member after 2 hours of light irradiation is denoted as Y5, a value of a magenta optical density of the ultraviolet-sensing member before the light irradiation is denoted as M4, and a value of a magenta optical density of the ultraviolet-sensing member after 2 hours from the light irradiation is denoted as M5,
all of a difference between C4 and C5, a difference between Y4 and Y5, and a difference between M4 and M5 are 0.15 or less.

10. The ultraviolet-sensing member according to claim 2,
wherein the photoactivator has a halomethyl-s-triazine structure.

11. The ultraviolet-sensing member according to claim 2,
wherein the color-forming agent has a spirolactone structure represented by Formula (I), 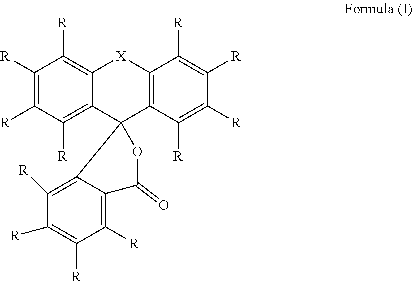
in Formula (I), X represents an oxygen atom, a sulfur atom, or —NR19-, where R19 represents a hydrogen atom, an alkyl group, or an aryl group, and R's each independently represent a hydrogen atom or a monovalent substituent.

12. An ultraviolet-sensing member,
wherein a difference between C1 and C2, a difference between Y1 and Y2, a difference between M1 and M2, and a rate of change in optical density satisfy the requirements of claim 5,
the ultraviolet-sensing member comprises a sensitizer having an absorption maximal wavelength in a range of 200 to 230 nm, a photoactivator, and a color-forming agent, and
the sensitizer has a naphthalene structure.

13. The ultraviolet-sensing member according to claim 2,
wherein the ultraviolet-sensing member is a sheet-like ultraviolet-sensing member.

14. An ultraviolet-sensing kit comprising:
the ultraviolet-sensing member according to claim 2.

15. An ultraviolet-sensing kit comprising:
the ultraviolet-sensing member according to claim 2,
wherein, in a case where the ultraviolet-sensing member is irradiated with light until an irradiation amount of light having a wavelength of 313 nm reaches 9 mJ/cm²,
using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and
a value of a cyan optical density of the ultraviolet-sensing member before the light irradiation is denoted as C4, a value of a cyan optical density of the ultraviolet-sensing member after 2 hours from the light irradiation is denoted as C5, a value of a yellow optical density of the ultraviolet-sensing member before the light irradiation is denoted as Y4, a value of a yellow optical density of the ultraviolet-sensing member after 2 hours of light irradiation is denoted as Y5, a value of a magenta optical density of the ultraviolet-sensing member before the light irradiation is denoted as M4, and a value of a magenta optical density of the ultraviolet-sensing member after 2 hours from the light irradiation is denoted as M5,
all of a difference between C4 and C5, a difference between Y4 and Y5, and a difference between M4 and M5 are 0.15 or less.

16. The ultraviolet-sensing member according to claim 3,
wherein the color-forming agent has a spirolactone structure represented by Formula (I), 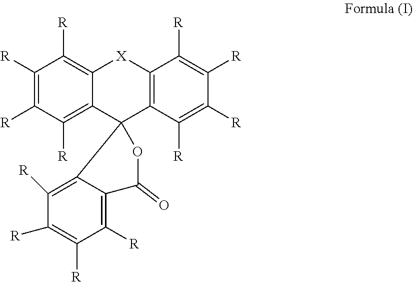
in Formula (I), X represents an oxygen atom, a sulfur atom, or —NR19-, where R19 represents a hydrogen atom, an alkyl group, or an aryl group, and R's each independently represent a hydrogen atom or a monovalent substituent.

17. An ultraviolet-sensing member,
wherein a difference between C1 and C2, a difference between Y1 and Y2, a difference between M1 and M2, and a rate of change in optical density satisfy the requirements of claim 5,
the ultraviolet-sensing member comprises a sensitizer having an absorption maximal wavelength in a range of 200 to 230 nm, a photoactivator, and a color-forming agent,
the sensitizer has a naphthalene structure, and
the photoactivator has a halomethyl-s-triazine structure.

18. The ultraviolet-sensing member according to claim 3,
wherein the ultraviolet-sensing member is a sheet-like ultraviolet-sensing member.

19. An ultraviolet-sensing kit comprising:
the ultraviolet-sensing member according to claim 3.

20. An ultraviolet-sensing kit comprising:
the ultraviolet-sensing member according to claim 3,
wherein, in a case where the ultraviolet-sensing member is irradiated with light until an irradiation amount of light having a wavelength of 313 nm reaches 9 mJ/cm²,
using a spectrocolorimeter X-Rite (manufactured by X-Rite), values of yellow optical densities, magenta optical densities, and cyan optical densities are measured with the ultraviolet-sensing member before the light irradiation and the ultraviolet-sensing member after the light irradiation, and
a value of a cyan optical density of the ultraviolet-sensing member before the light irradiation is denoted as C4, a value of a cyan optical density of the ultraviolet-sensing member after 2 hours from the light irradiation is denoted as C5, a value of a yellow optical density of the ultraviolet-sensing member before the light irradiation is denoted as Y4, a value of a yellow optical density of the ultraviolet-sensing member after 2 hours of light irradiation is denoted as Y5, a value of a magenta optical density of the ultraviolet-sensing member before the light irradiation is denoted as M4, and a value of a magenta optical density of the ultraviolet-sensing member after 2 hours from the light irradiation is denoted as M5,
all of a difference between C4 and C5, a difference between Y4 and Y5, and a difference between M4 and M5 are 0.15 or less.
